SEQUENCE LISTING (26 sequences)

SEQ ID NO: 1 - protein, 247 aa, synthetic construct
  source 1..247: note = cPD-L1_12C10E4_dK light chain of the cPD-L1 12C10E4 antibody
MESDTLLLWVLLLWVPGSAGDIVLTQSPASLAVSLGQRATISCRASESVEYYGTSLMQWYQQKPGQPPKLLIFAASNVKSGVPARFSGSGSGTDFSLNIHPVEEDDIAMYFCQQSGKVPHTFGGGTKLEIKRSMEIKRTDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 2 - DNA, 744 bp, synthetic construct
  source 1..744: mol_type = other DNA; note = cPD-L1_12C10E4_dK light chain of the cPD-L1 12C10E4 antibody
atggagagcgacaccctcctgctgtgggtgctgctactgtgggttcctgggagcgcgggagacatcgtgctgacacagagccctgcaagcctggccgtgagcctgggacagagagccaccatcagctgcagagcaagcgagagcgtggagtactacggcacaagcctgatgcagtggtatcagcagaagcctggacagcctcctaagctgctgatcttcgccgcaagcaacgtgaagagcggcgtgcctgctagattcagcggcagcggcagcggcaccgacttcagcctgaacatccaccctgtggaggaggacgacatcgccatgtacttctgtcagcagagcggcaaggtgcctcacaccttcggcggcggcaccaagctggagatcaagagatccatggaaatcaaacgtacggatgcccagccagccgtctatttgttccaaccatctccagaccagttacacacaggaagtgcctctgttgtgtgtttgctgaatagcttctaccccaaagacatcaatgtcaagtggaaagtggatggtgtcatccaagacacaggcatccaggaaagtgtcacagagcaggacaaggacagtacctacagcctcagcagcaccctgacgatgtccagtactgagtacctaagtcatgagttgtactcctgtgagatcactcacaagagcctgccctccaccctcatcaagagcttccaaaggagcgagtgtcagagagtggactaa

SEQ ID NO: 3 - protein, 472 aa, synthetic construct
  source 1..472: note = cPD-L1_12C10E4_dIgG2_heavy chain of the cPD-L1 12C10E4 antibody
MAVLGLLFCLVTLPSCVLSQVQLKQSGPGLVQPSQSLSITCTISGFSLTSFGVHWVRQSPGKGLEWLGVIWSGGSTDYNAAFTSRLSINKDNSKSQVFFKMNSLQADDTAIYYCARGGGPDWYFDVWGTGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 4 - DNA, 1419 bp, synthetic construct
  source 1..1419: mol_type = other DNA; note = cPD-L1_12C10E4_dIgG2_heavy chain of the cPD-L1 12C10E4 antibody
atggccgtgctgggcctgctgttctgcctggtgaccctgcctagctgcgtgctgagccaagtgcagctgaagcagagcggccctggcctggtgcagccttctcagagcctgagcatcacctgcaccatcagcggcttcagcctgacaagcttcggcgtgcactgggtgagacagagccctggcaagggcctggagtggctgggcgtgatctggagcggcggcagcaccgactacaacgccgccttcacaagcagactgagcatcaacaaggacaacagcaagagccaagtgttcttcaagatgaacagcctgcaagccgacgacaccgccatctactactgcgctagaggcggcggccctgactggtacttcgacgtgtggggcaccggcaccaccgtgaccgtgagcagcgctagcaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggctctactccctcagcagcatggtgacagtgccctccagcaggtggcccagcgagaccttcacctgcaacgtggcccacccggccagcaaaactaaagtagacaagccagtgcccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaggcccatcaacccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacagaaatccctctcccattctccgggtaaatga

SEQ ID NO: 5 - protein, 243 aa, synthetic construct
  source 1..243: note = cPD-L1_3C8D3_dK_light chain of the cPD-L1 3C chimeric antibody clone
METHSQVFVYMLLWLSGVEGDIVMTQSHKFMSTSVGDRVSITCKVSQDVGTAVAWYQQKPGQCPKRLIYWTSTRHTGVPDRFTGSGSGTDFTLTISNVQSEDLVDYFCQQYSSYPLTFGGGTKLELKRSMEIKRTDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 6 - DNA, 732 bp, synthetic construct
  source 1..732: mol_type = other DNA; note = cPD-L1_3C8D3_dK_light chain of the cPD-L1 3C chimeric antibody clone
atggagacacattctcaggtctttgtatacatgttgctgtggttgtctggtgttgaaggagacattgtgatgacccagtctcacaaattcatgtccacatcagtaggagacagggtcagcatcacctgcaaggttagtcaggatgtgggtaccgctgtagcctggtatcaacagaaaccagggcaatgtcccaaaagactgatttactggacatccacccggcacactggagtccctgatcgcttcacaggcagtggatctgggacagatttcactctcaccattagcaatgtgcagtctgaagacttggtagattatttctgtcagcaatatagcagttatcctctcacgttcggtggtgggaccaagctggagctgaaacggtccatggaaatcaaacgtacggatgcccagccagccgtctatttgttccaaccatctccagaccagttacacacaggaagtgcctctgttgtgtgtttgctgaatagcttctaccccaaagacatcaatgtcaagtggaaagtggatggtgtcatccaagacacaggcatccaggaaagtgtcacagagcaggacaaggacagtacctacagcctcagcagcaccctgacgatgtccagtactgagtacctaagtcatgagttgtactcctgtgagatcactcacaagagcctgccctccaccctcatcaagagcttccaaaggagcgagtgtcagagagtggactaa

SEQ ID NO: 7 - protein, 474 aa, synthetic construct
  source 1..474: note = cPD-L1_3C8D3_dIgG2_heavy chain of the cPD-L1 3C chimeric antibody clone
MGWSWIFLFLLSGTAGVLSEVQLQQSGPELVKPGASVKMSCKASGYTFTDYVLHWVRQTNGKSLEWIGEINPSNGDTYYNQKFKGKATLTVDTSSSTAYMQLTTLTSEDSAVYYCARSDYSNYVGFAYWGQGTLVTVSAASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 8 - DNA, 1425 bp, synthetic construct
  source 1..1425: mol_type = other DNA; note = cPD-L1_3C8D3_dIgG2_heavy chain of the cPD-L1 3C chimeric antibody clone
atgggatggagctggatctttctctttctcttgtcaggaactgcaggtgtcctctctgaagtccagctgcaacagtctggacctgagctggtgaagcctggggcttcagtgaagatgtcctgcaaggcttctggatacacattcactgactatgttttgcactgggtgaggcagaccaatggaaagagccttgagtggattggagaaattaatcctagcaatggtgatacttactacaaccagaagttcaagggcaaggccacattgactgtagacacatcctccagcacagcctacatgcagctcaccaccctgacatctgaggactctgcagtctattactgtgcacgatcggattatagtaactacgtaggatttgcttactggggccaagggactctggtcactgtctctgcagctagcaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggctctactccctcagcagcatggtgacagtgccctccagcaggtggcccagcgagaccttcacctgcaacgtggcccacccggccagcaaaactaaagtagacaagccagtgcccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaggcccatcaacccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacagaaatccctctcccattctccgggtaaatga

SEQ ID NO: 9 - protein, 10 aa, synthetic construct
  source 1..10: note = a CDR1
ESVEYYGTSL

SEQ ID NO: 10 - protein, 11 aa, synthetic construct
  source 1..11: note = a CDR3
CQQSGKVPHTF

SEQ ID NO: 11 - protein, 12 aa, synthetic construct
  source 1..12: note = a CDR3
ARGGGPDWYFDV

SEQ ID NO: 12 - protein, 11 aa, synthetic construct [sequence omitted]

SEQ ID NO: 13 - protein, 15 aa, synthetic construct
  source 1..15: note = a CDR3
CARSDYSNYVGFAYW

SEQ ID NO: 14 - protein, 20 aa, synthetic construct
  source 1..20: note = a portion of the sequence for a cPD-L1 12C chi antibody clone
GDTFICAVMHEALHNHYTQK

SEQ ID NO: 15 - protein, 8 aa, synthetic construct
  source 1..8: note = a CDR1
GFSLTSFG

SEQ ID NO: 16 - protein, 7 aa, synthetic construct
  source 1..7: note = a CDR2
IWSGGST

SEQ ID NO: 17 - protein, 6 aa, synthetic construct
  source 1..6: note = a CDR1
QDVGTA

SEQ ID NO: 18 - protein, 8 aa, synthetic construct
  source 1..8: note = a CDR1
GYTFTDYV

SEQ ID NO: 19 - protein, 8 aa, synthetic construct
  source 1..8: note = a CDR2
INPSNGDT

SEQ ID NO: 20 - RNA, 23 nt, synthetic construct
  source 1..23: mol_type = other RNA; note = pre-assembled guide RNA related to full length of canine CD274 cDNA (NM_001291972)
cagcaaatatcctcatgttttgg

SEQ ID NO: 21 - RNA, 20 nt, synthetic construct
  source 1..20: mol_type = other RNA; note = forward primer of primer set 1
ccacttggttctacatggct

SEQ ID NO: 22 - RNA, 21 nt, synthetic construct
  source 1..21: mol_type = other RNA; note = reverse primer of primer set 1
cctcagcctgacacattagtt

SEQ ID NO: 23 - RNA, 21 nt, synthetic construct
  source 1..21: mol_type = other RNA; note = forward primer of primer set 2
cctgtcacctctgaacatgaa

SEQ ID NO: 24 - RNA, 21 nt, synthetic construct
  source 1..21: mol_type = other RNA; note = reverse primer of primer set 2
ggactaagctctaggttgtcc

SEQ ID NO: 25 - RNA, 22 nt, synthetic construct
  source 1..22: mol_type = other RNA; note = forward primer of primer set 3
gactggcttttagggcttatgt

SEQ ID NO: 26 - RNA, 23 nt, synthetic construct
  source 1..23: mol_type = other RNA; note = reverse primer of primer set 3
acaccccacaaattacttccatt

(19) United States
(12) Patent Application Publication
(10) Pub. No.: US 2026/0193355 A1
(43) Pub. Date: Jul. 9, 2026

(54) CANINE PD-L1 ANTIBODY, ANTIGEN BINDING FRAGMENTS THEREOF, AND METHODS OF USE THEREOF

(71) Applicant: PURDUE RESEARCH FOUNDATION, West Lafayette, IN (US)

(72) Inventors: Deborah KNAPP, West Lafayette, IN (US); Deepika DHAWAN, West Lafayette, IN (US); Seung-Oe LIM, West Lafayette, IN (US); Perry KIRKHAM, Lafayette, IN (US)

(21) Appl. No.: 19/129,848

(22) PCT Filed: Nov. 14, 2023
(86) PCT No.: PCT/US2023/079712

§ 371 Date: May 14, 2025

Related U.S. Application Data
(60) Provisional application No. 63/383,909, filed on Nov. 15, 2022.

(51) Int. Cl. C07K 16/28; A61K 39/00; A61P 35/00

(52) U.S. Cl. CPC C07K 16/2827; A61P 35/00; A61K 2039/505; A61K 2039/545; C07K 2317/24; C07K 2317/565; C07K 2317/734; C07K 2317/92; C07K 2317/94

(57) ABSTRACT

Caninized antibodies and antigen binding fragments thereof are provided that bind programmed death ligand 1 in a canine subject. Methods of treating cancer in a canine subject using the caninized antibodies and/or antigen binding fragments are also provided, as are methods for predicting and modeling anti-cancer activity of a test compound in a subject.

### PRIORITY

This patent application is related to and claims the priority benefit of U.S. Provisional Patent Application No. 63/383,909 filed Nov. 15, 2022. The content of the foregoing application is hereby incorporated by reference in its entirety into this disclosure.

### TECHNICAL FIELD

This disclosure relates to anti-canine programmed death-ligand 1 (PD-L1) antibodies, antigen binding fragments thereof, complementarity determining regions (CDRs) thereof, and caninized antibodies against canine PD-L1. Methods of predicting and modeling anti-cancer activity of a test compound in a subject using the antibodies, CDRs, and/or antigen binding fragments thereof are also provided.

### BACKGROUND

In a normal healthy system, immune checkpoints are surface proteins that are present to check and thwart any overstimulation of immune response. In other words, their role is to prevent an immune response from being so strong that it destroys healthy cells in the body. In the case of cancer, when a T cell checkpoint protein (e.g., programmed cell death protein 1 (PD-1)) engages with its binding protein on a tumor cell (programmed death-ligand 1 (PD-L1)), it sends an “off” signal to the T cells and suppresses the anti-tumor immune response. In this manner, tumor killer cells within the immune system cannot kill the tumor because their method of attack is blocked by those checkpoints on the tumor itself.

Immune checkpoint blockade therapy employs immune checkpoint inhibitors and functions by inhibiting the checkpoint proteins from binding with their ligands on tumor cells. PD-1, cytotoxic T-lymphocyte-associated protein 4 (CTLA-4), lymphocyte activating 3 (LAG3), T cell immunoglobulin and mucin domain-containing protein 3 (TIM3), T cell immunoreceptor with Ig and ITIM domains (TIGIT) and V-domain Ig suppressor of T cell activation (VISTA) are some of the examples of checkpoint proteins. Immune checkpoint blockade therapy is one of the most promising forms of cancer immunotherapy and has been successful in multiple cancer types, including invasive urinary bladder cancer. In particular, the PD-1/PD-L1 pathway blockade using anti-PD-1 or anti-PD-L1 antibodies has elicited durable clinical responses in cancer patients, for example, by normalizing the imbalanced anti-tumor immunity.

Given the promising and durable clinical responses, the U.S. Food and Drug Administration (FDA) has approved three PD-1 antibodies (Pembrolizumab (Keytruda®) and Nivolumab (Opdivo®), and cemiplimab) and three PD-L1 antibodies (atezolizumab, avelumab, and durvalumab) for multiple types of cancer in humans. While this milestone suggests the promise of cancer immunotherapy treatment, PD-1/PD-L1 blockade therapy in cancer is not satisfactory at present due to the limited response rates (10-40%). Therefore, new immunotherapeutic strategies to improve the therapeutic efficacy of the current PD-1/PD-L1 blockade are urgently needed.

Strategies to improve PD-1/PD-L1 blockade therapies in bladder and other cancers can include 1) identifying host factors including genetics, immune state, and molecular subtype that drive response, 2) assessing biomarkers and combinations of biomarkers to predict response and personalize therapy, 3) developing better tools to monitor immune effects, and 4) selecting combination drug approaches/regimens to address multiple “defects” in the immune response in addition to PD-1/PD-L1 blockade. Relevant pre-clinical animal models are typically used to develop these strategies and test combination approaches. Factors that are likely to affect the PD-1/PD-L1 axis and, thus, factors that can be represented in animal models, include aggressive and metastatic cancer behavior, tumor heterogeneity, mutational landscape, genetic and epigenetic crosstalk, cancer molecular subtypes, immune cell responsiveness, and innate and acquired mechanisms of drug resistance. While rodent models, including carcinogen-induced, engraftment, and genetically engineered models, are instrumental in cancer research, rodent models do not possess the collective features that are critical to studying emerging therapies within and across molecular subtypes in bladder cancer, and predicting therapeutic success or failure in humans. What is needed is an animal model that can be used to optimize checkpoint inhibitor treatment in humans.

In addition, many rodent data are not predictive for canines, especially with respect to immune checkpoint blockade therapy. Many cancers remain untreatable in canines, and therapeutic approaches effective for one cancer often fail in another. Thus, there is also a need to treat certain forms of cancer in canines.

### SUMMARY

A caninized antibody or antigen binding fragment thereof that binds programmed death ligand 1 (PD-L1) in a canine subject is provided. In certain embodiments, the antibody or antigen binding fragment is encoded by the nucleotide sequence comprises at least 80% sequence identity to SEQ ID NOS: 2 and/or 4 or SEQ ID NOS: 6 and/or 8 (e.g., comprises SEQ ID NOS: 2 and/or 4 or SEQ ID NOS: 6 and/or 8).

The antibody or antigen binding fragment can comprise one or more complementarity determining regions (CDRs) comprising AAS, SEQ ID NO: 9, and/or SEQ ID NO: 10. The antibody or antigen binding fragment can comprise CDRs, each CDR comprising at least 80% sequence identity to SEQ ID NO: 17, WTS, and/or SEQ ID NO: 12 (e.g., comprising SEQ ID NO: 17, WTS, and/or SEQ ID NO: 12). The antibody or antigen binding fragment can comprise CDRs independently comprising at least 80% sequence identity to SEQ ID NO: 15, SEQ ID NO: 16, and/or SEQ ID NO: 11 (e.g., comprising SEQ ID NO: 15, SEQ ID NO: 16, and/or SEQ ID NO: 11). The antibody or antigen binding fragment can comprise CDRs independently comprising at least 80% sequence identity to SEQ ID NO: 18, SEQ ID NO: 19, and/or SEQ ID NO: 13 (e.g., comprising SEQ ID NO: 18, SEQ ID NO: 19, and/or SEQ ID NO: 13).

In certain embodiments, the antibody or antigen binding fragment is completely or incompletely caninized.

The antibody or antigen binding fragment can be a chimeric form of a caninized antibody or antigen binding fragment. The antibody or antigen binding fragment thereof can, in certain embodiments, bind canine PD-L1 with specificity. In certain embodiments, the antibody or antigen binding fragment comprises a caninized murine PD-1 antibody or antigen binding fragment thereof.

Methods for treating cancer in a canine subject are also provided. Such a method can comprise administering to a canine subject a therapeutically effective amount of a caninized antibody or antigen binding fragment thereof that binds PD-L1 and inhibits a PD-L1/PD-1 interaction in the subject.

The caninized antibody or antigen binding fragment thereof of the method can comprise any of the caninized antibodies or antigen binding fragments thereof described herein. In certain embodiments, for example, the caninized antibody or antigen binding fragment thereof is encoded by a nucleotide sequence comprising at least 80% sequence identity to SEQ ID NOS: 2 and/or 4 or SEQ ID NOS: 6 and/or 8. The caninized antibody or antigen binding fragment can comprise one or more CDRs independently comprising at least 80% sequence identity to AAS, SEQ ID NO: 9, and/or SEQ ID NO: 10. The caninized antibody or antigen binding fragment can comprise one or more CDRs independently comprising at least 80% sequence identity to SEQ ID NO: 18, WTS, and/or SEQ ID NO: 12. The caninized antibody or antigen binding fragment can comprise one or more CDRs independently comprising at least 80% sequence identity to SEQ ID NO: 18, SEQ ID NO: 19, and/or SEQ ID NO: 13.

In certain embodiments of the method, the caninized antibody or antigen binding fragment is encoded by the nucleotide sequence comprising SEQ ID NOS: 2 and/or 4 or SEQ ID NOS: 6 and/or 8. In certain embodiments of the method, the caninized antibody or antigen binding fragment comprises one or more CDRs comprising AAS, SEQ ID NO: 9, and/or SEQ ID NO: 10. In certain embodiments of the method, the caninized antibody or antigen binding fragment comprises one or more CDRs comprising SEQ ID NO: 17, WTS, and/or SEQ ID NO: 12. In certain embodiments of the method, the caninized antibody or antigen binding fragment comprises one or more CDRs comprising SEQ ID NO: 15, SEQ ID NO: 16, and/or SEQ ID NO: 11. The caninized antibody or antigen binding fragment of the method can comprise one or more CDRs comprising SEQ ID NO: 18, SEQ ID NO: 19, and/or SEQ ID NO: 13.

In certain embodiments, the therapeutically effective amount is from about 2 mg/kg of subject body weight (such as, for example, 2 mg/kg of subject body weight) to about 5 mg/kg of subject body weight (such as, for example, 5 mg/kg of subject body weight). The therapeutically effective amount can be 2 mg/kg of subject body weight. The therapeutically effective amount can be 5 mg/kg of subject body weight. The therapeutically effective amount can be from about 2.5 mg/kg of subject body weight (such as, for example, 2.5 mg/kg of subject body weight) to about 4.5 mg/kg of subject body weight (such as, for example, 4.5 mg/kg of subject body weight). The therapeutically effective amount can be from about 3.0 mg/kg of subject body weight (such as, for example, 3.0 mg/kg of subject body weight) to about 4.0 mg/kg of subject body weight (such as, for example, 4.0 mg/kg of subject body weight). The therapeutically effective amount can be from about 2.5 mg/kg of subject body weight (such as, for example, 3.0 mg/kg of subject body weight) to about 3.5 mg/kg of subject body weight (such as, for example, 3.5 mg/kg of subject body weight). The therapeutically effective amount can be 2 mg/kg of subject body weight. The therapeutically effective amount can be 5 mg/kg of subject body weight. The therapeutically effective amount can be from about 5.0 mg/kg of subject body weight (such as, for example, 5.0 mg/kg of subject body weight) to about 10.0 mg/kg of subject body weight (such as, for example, 10.0 mg/kg of subject body weight). The therapeutically effective amount can be from about 5.5 mg/kg of subject body weight (such as, for example, 5.5 mg/kg of subject body weight) to about 9.5 mg/kg of subject body weight (such as, for example, 9.5 mg/kg of subject body weight). The therapeutically effective amount can be from about 6.0 mg/kg of subject body weight (such as, for example, 6.0 mg/kg of subject body weight) to about 9.0 mg/kg of subject body weight (such as, for example, 9.0 mg/kg of subject body weight). The therapeutically effective amount can be from about 6.5 mg/kg of subject body weight (such as, for example, 6.5 mg/kg of subject body weight) to about 8.5 mg/kg of subject body weight (such as, for example, 8.5 mg/kg of subject body weight). The therapeutically effective amount can be from about 7.0 mg/kg of subject body weight (such as, for example, 7.0 mg/kg of subject body weight) to about 8.0 mg/kg of subject body weight (such as, for example, 8.0 mg/kg of subject body weight). The therapeutically effective amount can be from about 7.5 mg/kg of subject body weight (such as, for example, 7.5 mg/kg of subject body weight) to about 7.8 mg/kg of subject body weight (such as, for example, 7.8 mg/kg of subject body weight). The ranges specified in this paragraph are inclusive of the end points stated and all 0.1 mg/kg increments contained within the specified ranges.

The cancer can be invasive urothelial carcinoma.

In certain embodiments of the method, the antibody or antigen binding fragment thereof is formulated into a pharmaceutical composition.

Pharmaceutical compositions are also provided. In certain embodiments, the pharmaceutical composition comprises any of the caninized antibodies or antigen binding fragments thereof described herein and a pharmaceutically acceptable excipient.

Methods for predicting and modeling anti-cancer activity of a test compound in a subject (e.g., having cancer) are also provided. In certain embodiments, such a method comprises: generating a population of caninized PD-L1 mice that express canine PD-L1 on a cell surface; evaluating toxicity risk and/or efficacy in treating a cancer of a set of test compounds in the population of caninized PD-L1 mice; and selecting one or more test compounds of the set that satisfy established toxicity risk and/or efficacy criteria.

Generating a population of caninized PD-L1 mice can further comprise replacing a mouse cd274 gene in a population of mice with a canine PD-L1 gene using CRISPR.

The method can further comprise evaluating the toxicity risk and/or efficacy of the one or more selected compounds in treating a cancer in a human or canine cohort. In certain embodiments, the method can further comprise evaluating oral bioavailability, adsorption, distribution, metabolism and excretion (ADME) values of the set of test compounds in the population of caninized PD-L1 mice. In certain embodiments, the method further comprises evaluating oral bioavailability and ADME values of the selected one or more test compounds in the population of caninized PD-L1 mice.

In certain embodiments, the set of test compounds comprises at least a compound that inhibits PD-L1/PD-1 interaction in the subject.

CDRs of an antibody or antigen binding fragment are also provided. The CDR can comprise at least 80% sequence identity to AAS, SEQ ID NO: 9, and/or SEQ ID NO: 10. The CDR of an antibody or antigen binding fragment can comprise at least 80% sequence identity to SEQ ID NO: 18, WTS, and/or SEQ ID NO: 12. The CDR of an antibody or antigen binding fragment can comprise at least 80% sequence identity to SEQ ID NO: 16, SEQ ID NO: 17, and/or SEQ ID NO: 11. The CDR can comprise at least 80% sequence identity to SEQ ID NO: 18, SEQ ID NO: 19, and/or SEQ ID NO: 13. The CDR can comprise at least 80% sequence identity to SEQ ID NO: 17, WTS, and/or SEQ ID NO: 12. The CDR of an antibody or antigen binding fragment can comprise at least 80% sequence identity to SEQ ID NO: 16, SEQ ID NO: 17, and/or SEQ ID NO: 11. The CDR of an antibody or antigen binding fragment can comprise at least 80% sequence identity to SEQ ID NO: 18, SEQ ID NO: 19, and/or SEQ ID NO: 13.

In certain embodiments, a use of a caninized antibody or antigen binding fragment of the present disclosure, a pharmaceutical composition described herein, or one or more of the CDRs described herein is provided, such use in the preparation of a medicament for treating cancer in a canine subject.

### DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein:

FIG. 1 shows a schematic flow chart representing the production and validation of canine PD-L1 antibody used herein, with (A) Immunization of antigen, canine PD-L1 protein (cPD-L1) injection, (B) Establishment of hybridomas (over 2,000 clones), (C) cPD-L1 specific antibody (Ab) selection by the live cell-based Ab binding assay, (D) Therapeutic Ab selection by cPD-1/PD-L1 blockade assay, (E) In vivo validation of therapeutic efficacy of cPD-L1 antibodies in the mice that have only canine PD-L1 (i.e. caninized PD-L1 mice) and PD-1, (F) Production of the cPD-L1 chimeric Ab (mouse/canine), (G) Validation of the cPD-L1 chimeric Ab, and (H) Initial safety profile and a PK assay in dog.

FIG. 2A is a schematic diagram of the canine programmed death-ligand 1 (cPD-L1) antibody binding assay.

FIG. 2B is a schematic diagram of the cPD-L1/canine programmed death protein 1 (cPD-1) blockade assay.

FIG. 2C is a kinetic graph showing quantitative binding of programmed death-ligand 1 (PD-L1) antibodies on BT549 cells expressing cPD-L1 at every 3-hour time point with the positive clones highlighted in red.

FIG. 2D are representative images (at 18 hours) of cPD-L1 antibody binding, with green fluorescent merged images of cPD-L1 expressing cells shown.

FIG. 2E is a kinetic graph showing quantitative binding of PD-1 protein on BT549 cells expressing cPD-L1 at every 3-hour time points after the addition of cPD-L1 antibodies. The positive clones that blocked the interaction of PD-L1/PD-1 proteins are highlighted in red.

FIG. 2F are representative images (at 18 hours) of the cPD-L1 blockade, with green fluorescent merged images of cPD-L1 expressing cells shown. Note the lack of fluorescence due to the antibody binding to PD-L1 and blocking the interaction with cPD-1.

FIG. 3A is a schematic depicting the knock-in strategy of the mice containing the canine PD-L1 and PD-1 molecules (c57BL/c background).

FIG. 3B is a validation of cPD-L1 protein expression in the MB49^{cPD-L1} cells, showing flow cytometric analysis of membrane-located mPD-L1 and cPD-L1 protein in MB49 cells expressing cPD-L1 (MB49^{cPD-L1}) or MB49 parental cells.

FIG. 3C shows immunofluorescence staining and protein expression patterns of mPD-L1 and cPD-L1 in MB49 or MB49^{cPD-L1} tumor masses from the caninized PD-L1 mice (DAPI used for nuclear counterstaining; scale bar 100 μM).

FIG. 3D shows a graph indicative of the interaction of canine PD-1 (cPD-1) or mouse PD-1 (mPD-1) protein with cPD-L1 or mouse PD-L1 (mPD-L1) protein with or without canine PD-L1 antibody (12C). Histidine (His)-tagged canine or mPD-L1 protein was immobilized on the nickel-nitriloacetic acid (Ni-NTA) 96-well plate and horseradish peroxidase (HRP)-conjugated anti-human IgG Fc-specific secondary with mPD-1-hFc or cPD-1-hFc protein was added. OD₄₅₀ was measured to quantify the amount of bound PD-1 protein.

FIG. 3E shows the binding of cPD-L1 antibodies, 12C and 3C, with human PD-L1 (hPD-L1), mPD-L1, and cPD-L1 proteins. His-tagged human PD-L1, mPD-L1, or cPD-L1 protein was immobilized on the Ni-NTA 96-well plate, and anti-cPD-L1 Abs, 12C or 3C, with HRP-conjugated anti-canine IgG-specific secondary was added. OD₄₅₀ was measured to quantify the amount of bound PD-L1 antibodies (Ab=antibody).

FIG. 3F shows data related to tumor growth of MB49^{cPD-L1} in the caninized PD-L1 mice treated with cPD-L1 antibody, 12C or 3C. The IgG isotype of 12C and 3C antibodies is mouse IgG1, which is equivalent to human IgG4. Tumors were measured at the indicated time points and dissected at the endpoint (n=8 per group).

FIG. 3G shows representative images of immunofluorescence staining of the protein expression pattern of CD8 and granzyme B in MB49 tumor masses from IgG-, 12C-, or 3C-treated mice. DAPI was used for nuclear counterstaining. Scale bar, 100 μm.

FIGS. 3H and 3I show quantification of CD8 (FIG. 3H) and granzyme B (FIG. 3I) in the immunofluorescence staining and protein expression patterns using Gen5 software (BioTek, Winooski, VT). n=10.

FIGS. 3J and 3K are graphs representative of the effect of treatment on the PD-L1 mice, with mice kidney (FIG. 3J) and liver (FIG. 3K) functions measured at the end of the experiments. ALT, alanine aminotransferase. Treatment with the PD-L1 antibody did not affect kidney function (serum creatinine) or liver enzyme activity (ALT=alanine aminotransferase), measured in blood collected at the end of the experiment.

FIGS. 4A-4F show the quality attributes of purified anti-cPD-L1, 1210E4 (12C) chimeric antibody, with FIG. 4A showing a schematic of the cPD-L1, 12C10E4 chimeric antibody expression construct, pTRIOZ-cIgG2-cPD-L1 12C10E4; FIG. 4B showing a sodium dodecyl sulfate-polyacrylamide gel electrophoresis (SDS-PAGE) analysis of 12C chimeric antibody purity under nonreducing and reducing (2-mercaptoethanol) conditions (HC, heavy chain; LC, light chain; SM, protein size marker); FIG. 4C showing an isoelectric focusing (IEF) analysis of 12C chimeric antibody (standard, pI standard); FIG. 4D showing peptide mapping analysis of the cPD-L1 12C chimeric antibody (SEQ ID NO: 14); the 12C chimeric antibody was enzymatically digested with trypsin on S-trap micro columns from Protifi after reduction and alkylation. Peptides were then separated and analyzed by reversed-phase liquid chromatography tandem mass spectrometry (RP-LC/MS-MS). The resultant mass spectrometric data were analyzed using the PEAK PTM workflow int eh PEAKS X PRO Studio 10.6 software package from Bioinformatics Solutions Inc. to map the detected MS1 and MS2 ions to the amino acid sequence of the antibody. The sequence coverage of heavy and light chains was 100% (453 of 453 amino acids) and 98.2% (223 of 227 amino acids), respectively); FIG. 4E showing size exclusion chromatography (SEC) analysis of 12C chimeric antibody (standard, SEC standard); and FIG. 4F showing a matrix-assisted laser desorption/ionization (MALDI)-mass spectrometry (MS) profile of permethylated N-glycans released from PNGase F-treated 12C chimeric antibody, with the masses of indicated glycan species representing the [M+Na+] values.

FIG. 5A shows 12C antibody binding on the BT549^{cPD-L1} cells.

FIG. 5B shows a flow cytometer analysis by 12C chimeric antibody in the BT549^{cPD-L1} cells, with cIgG serving as a negative control.

FIG. 5C shows 12C chimeric antibody binding to cPD-L1 and cPD-L2.

FIG. 5D shows a binding affinity (K_{D}) analysis of 12C chimeric antibody by Octet.

FIG. 5E shows EC₅₀ of 12C chimeric antibody, 12C10E4. EC₅₀=0.419 μg/ml, with the bound cPD-1 proteins quantified by measuring green fluorescence at the IncuCyte® S3 Live-Cell Analysis System (Sartorius AG, Gottingen, Germany).

FIGS. 5F and 5G show images related to data from Canine IO Panel (NanoString) analyses, which were used to query changes in gene expression upon activation of canine peripheral blood mononuclear cells (cPBMCs) from three healthy pet dogs. The RNA from the resting and activated PBMCs was used for NanoString work. The Canine IO Panel was used to query the changes in approximately 700 genes. Groupwise analyses were conducted using “Rosalind.” There were 65 genes that were differentially expressed when comparing control PBMCs with activated PBMCs (P<0.05, FC>1.5) including 30 upregulated and 35 downregulated genes. In the heatmap, each column consists of data from one sample.

FIGS. 5H and 5I show data related to interferon gamma (IFNγ) and tumor necrosis factor alpha (TNFα) concentrations, respectively, in activated cPBMCs.

FIGS. 5J and 5K are flow cytometric analysis data of cPD-L1 protein expression on the K9TCC or the nuclear-restricted RFP-expressing K9TCC (K9TCC^{nRFP}) cells using the 123C chimeric Ab. The endogenous PD-L1 expression was stimulated by 50 ng/ml canine IFNγ for 12 hours. cIgG served as a negative control.

FIG. 5L shows data from a quantitative reverse transcription-polymerase chain reaction (RT-PCR) of cPD-L1 (CD274) mRNA expression in the K9TCC or K9TCCIRFP cells.

FIG. 5M is images and graphical data related to K9TCC cells that were co-cultured with cPBMCs that were activated with CD3 antibody (100 ng/mL) and interleukin-2 (IL-2) (10 ng/ml) at a ratio of 1 tumor cell: 15 cPBMCs, with the live tumor cell count at 72 hours shown in the bar graph (right). The 12C chimeric antibody enhanced the tumor cell killing.

FIG. 5N shows data related to the analysis of IFNγ concentrations in K9TCC cells and activated cPBMCs co-cultured medium with and without the 12C chimeric antibody treatment.

FIG. 6A shows a schematic diagram of enzyme-linked immunosorbent assay (ELISA) for pharmacokinetics analysis.

FIG. 6B is a graph of the concentration of the 12C chimeric antibody measured in the 2 mg/kg 12C antibody-treated dogs' serum.

FIG. 6C is a graph of the concentration of the 12C chimeric antibody measured in the 5 mg/kg 12C antibody-treated dogs' serum.

While the present disclosure is susceptible to various modifications and alternative forms, exemplary embodiments thereof are shown by way of example in the drawings and are herein described in detail.

### SEQUENCE LISTINGS

The sequences herein (SEQ ID NOS: 1-26) are also provided in computer readable form encoded in a file filed herewith and incorporated herein by reference. The information recorded in computer readable form is identical to the written sequence listings provided herein, pursuant to 37 C.F.R. § 1.821 (f).

SEQ ID NO: 1 is the amino acid sequence for the cPD-L1_12C10E4_dK light chain of the cPD-L1 12C10E4 antibody (deduced from the DNA sequence):

MESDTLLLWVLLLWVPGSAGDIVLTQSPASLAVSLGQRATISCRASESV
EYYGTSLMQWYQQKPGQPPKLLIFAASNVKSGVPARFSGSGSGTDFSLN
IHPVEEDDIAMYFCQQSGKVPHTFGGGTKLEIKRSMEIKRTDAQPAVYL
FQPSPDQLHTGSASVVCLLNSFYPKDINVKWKVDGVIQDTGIQESVTEQ
DKDSTYSLSSTLTMSSTEYLSHELYSCEITHKSLPSTLIKSFQRSECQR
VD*,

wherein, Underline, signal sequence; Italic, FR; Bold, CDR; Bold and Underlined, CL; *, Stop; CDR1: ESVEYYGTSL (SEQ ID NO: 9); CDR2: AAS; and CDR3: CQQSGKVPHTF (SEQ ID NO: 10).

SEQ ID NO: 2 is the nucleotide sequence for the cPD-L1_12C10E4_dK light chain of the cPD-L1 12C10E4 antibody (and encodes SEQ ID NO: 1) (codon optimized for CHO cell):

ATGGAGAGCGACACCCTCCTGCTGTGGGTGCTGCTACTGTGGGTTCCTG
GGAGCGCGGGAGACATCGTGCTGACACAGAGCCCTGCAAGCCTGGCCGT
GAGCCTGGGACAGAGAGCCACCATCAGCTGCAGAGCAAGCGAGAGCGTG
GATGCAGTGGTATCAGCAGAAGCCTGGACAGCCTCCTAAGCTGCTGATC
TGAGTACTACGGCACAAGCCTTCGCCGCAAGCAACGTGAAGAGCGGCGT
GCCTGCTAGATTCAGCGGCAGCGGCAGCGGCACCGACTTCAGCCTGAAC
ATCCACCCTGTGGAGGAGGACGACATCGCCATGTACTTCTGTCAGCAGA
GCGGCAAGGTGCCTCACACCTTCGGCGGCGGCACCAAGCTGGAGATCAA
GAGAtCCATGGAAATCAAACGTACGGATGCCCAGCCAGCCGTCTATTTG
TTCCAACCATCTCCAGACCAGTTACACACAGGAAGTGCCTCTGTTGTGT
GTTTGCTGAATAGCTTCTACCCCAAAGACATCAATGTCAAGTGGAAAGT
GGATGGTGTCATCCAAGACACAGGCATCCAGGAAAGTGTCACAGAGCAG
GACAAGGACAGTACCTACAGCCTCAGCAGCACCCTGACGATGTCCAGTA
CTGAGTACCTAAGTCATGAGTTGTACTCCTGTGAGATCACTCACAAGAG
CCTGCCCTCCACCCTCATCAAGAGCTTCCAAAGGAGCGAGTGTCAGAGA
GTGGACTAA.

SEQ ID NO: 3 is the amino acid sequence for the cPD-L1_12C10E4_dIgG2_heavy chain of the cPD-L1 12C10E4 antibody (deduced from the DNA sequence):

MAVLGLLFCLVTLPSCVLSQVQLKQSGPGLVQPSQSLSITCTISGFSLT
SFGVHWVRQSPGKGLEWLGVIWSGGSTDYNAAFTSRLSINKDNSKSQVF
FKMNSLQADDTAIYYCARGGGPDWYFDVWGTGTTVTVSSASTTAPSVFP
LAPSCGSTSGSTVALACLVSGYFPEPVTVSWNSGSLTSGVHTFPSVLQS
SGLYSLSSMVTVPSSRWPSETFTCNVAHPASKTKVDKPVPKRENGRVPR
PPDCPKCPAPEMLGGPSVFIFPPKPKDTLLIARTPEVTCVVVDLDPEDP
EVQISWFVDGKQMQTAKTQPREEQFNGTYRVVSVLPIGHQDWLKGKQFT
CKVNNKALPSPIERTISKARGQAHQPSVYVLPPSREELSKNTVSLTCLI
KDFFPPDIDVEWQSNGQQEPESKYRTTPPQLDEDGSYFLYSKLSVDKSR
WQRGDTFICAVMHEALHINHYTQKSLSHSPGK*;

wherein Underline, signal sequence; Italic, FR; Bold, CDR; Bold and Underlined, CH; *, Stop; CDR1: GFSLTSFG (SEQ ID NO: 15); CDR2: IWSGGST (SEQ ID NO: 16); and CDR3: ARGGGPDWYFDV (SEQ ID NO: 11).

SEQ ID NO: 4 is the nucleotide sequence for the cPD-L1_12C10E4_dIgG2_heavy chain of the cPD-L1 12C10E4 antibody (and encodes SEQ ID NO: 3):

ATGGCCGTGCTGGGCCTGCTGTTCTGCCTGGTGACCCTGCCTAGCTGCG
TGCTGAGCCAAGTGCAGCTGAAGCAGAGCGGCCCTGGCCTGGTGCAGCC
TTCTCAGAGCCTGAGCATCACCTGCACCATCAGCGGCTTCAGCCTGACA
AGCTTCGGCGTGCACTGGGTGAGACAGAGCCCTGGCAAGGGCCTGGAGT
GGCTGGGCGTGATCTGGAGCGGCGGCAGCACCGACTACAACGCCGCCTT
CACAAGCAGACTGAGCATCAACAAGGACAACAGCAAGAGCCAAGTGTTC
TTCAAGATGAACAGCCTGCAAGCCGACGACACCGCCATCTACTACTGCG
CTAGAGGCGGCGGCCCTGACTGGTACTTCGACGTGTGGGGCACCGGCAC
CACCGTGACCGTGAGCAGCGCTAGCACCACGGCCCCCTCGGTTTTCCCA
CTGGCCCCCAGCTGCGGGTCCACTTCCGGCTCCACGGTGGCCCTGGCCT
GCCTGGTGTCAGGCTACTTCCCCGAGCCTGTAACTGTGTCCTGGAATTC
CGGCTCCTTGACCAGCGGTGTGCACACCTTCCCGTCCGTCCTGCAGTCC
TCAGGGCTCTACTCCCTCAGCAGCATGGTGACAGTGCCCTCCAGCAGGT
GGCCCAGCGAGACCTTCACCTGCAACGTGGCCCACCCGGCCAGCAAAAC
TAAAGTAGACAAGCCAGTGCCCAAAAGAGAAAATGGAAGAGTTCCTCGC
CCACCTGATTGTCCCAAATGCCCAGCCCCTGAAATGCTGGGAGGGCCTT
CGGTCTTCATCTTTCCCCCGAAACCCAAGGACACCCTCTTGATTGCCCG
AACACCTGAGGTCACATGTGTGGTGGTGGATCTGGACCCAGAAGACCCT
GAGGTGCAGATCAGCTGGTTCGTGGACGGTAAGCAGATGCAAACAGCCA
AGACTCAGCCTCGTGAGGAGCAGTTCAATGGCACCTACCGTGTGGTCAG
TGTCCTCCCCATTGGGCACCAGGACTGGCTCAAGGGGAAGCAGTTCACG
TGCAAAGTCAACAACAAAGCCCTCCCATCCCCGATCGAGAGGACCATCT
CCAAGGCCAGAGGGCAGGCCCATCAACCCAGTGTGTATGTCCTGCCGCC
ATCCCGGGAGGAGTTGAGCAAGAACACAGTCAGCTTGACATGCCTGATC
AAAGACTTCTTCCCACCTGACATTGATGTGGAGTGGCAGAGCAATGGAC
AGCAGGAGCCTGAGAGCAAGTACCGCACGACCCCGCCCCAGCTGGACGA
GGACGGGTCCTACTTCCTGTACAGCAAGCTCTCTGTGGACAAGAGCCGC
TGGCAGCGGGGAGACACCTTCATATGTGCGGTGATGCATGAAGCTCTAC
ACAACCACTACACACAGAAATCCCTCTCCCATTCTCCGGGTAAATGA.

SEQ ID NO: 5 is the amino acid sequence for the cPD-L1_3C8D3_dK_light chain of the cPD-L1 3C chimeric antibody clone:

METHSQVFVYMLLWLSGVEGDIVMTQSHKFMSTSVGDRVSITCKVSQDV
GTAVAWYQQKPGQCPKRLIYWTSTRHTGVPDRFTGSGSGTDFTLTISNV
QSEDLVDYFCQQYSSYPLTFGGGTKLELKRSMEIKRTDAQPAVYLFQPS
PDQLHTGSASVVCLLNSFYPKDINVKWKVDGVIQDTGIQESVTEQDKDS
TYSLSSTLTMSSTEYLSHELYSCEITHKSLPSTLIKSFQRSECQRVD*,

wherein Underline, signal sequence; Italic, FR; Bold, CDR; Bold and Underlined, CL; *, Stop; CDR1: QDVGTA (SEQ ID NO: 17); CDR2: WTS; and CDR3: CQQYSSYPLTF (SEQ ID NO: 12).

SEQ ID NO: 6 is the nucleotide sequence for the cPD-L1_3C8D3_dK_light chain of the cPD-L1 3C chimeric antibody clone (and encodes SEQ ID NO: 5):

ATGGAGACACATTCTCAGGTCTTTGTATACATGTTGCTGTGGTTGTCTG
GTGTTGAAGGAGACATTGTGATGACCCAGTCTCACAAATTCATGTCCAC
ATCAGTAGGAGACAGGGTCAGCATCACCTGCAAGGTTAGTCAGGATGTG
GGTACCGCTGTAGCCTGGTATCAACAGAAACCAGGGCAATGTCCCAAAA
GACTGATTTACTGGACATCCACCCGGCACACTGGAGTCCCTGATCGCTT
CACAGGCAGTGGATCTGGGACAGATTTCACTCTCACCATTAGCAATGTG
CAGTCTGAAGACTTGGTAGATTATTTCTGTCAGCAATATAGCAGTTATC
CTCTCACGTTCGGTGGTGGGACCAAGCTGGAGCTGAAACGGTCCATGGA
AATCAAACGTACGGATGCCCAGCCAGCCGTCTATTTGTTCCAACCATCT
CCAGACCAGTTACACACAGGAAGTGCCTCTGTTGTGTGTTTGCTGAATA
GCTTCTACCCCAAAGACATCAATGTCAAGTGGAAAGTGGATGGTGTCAT
CCAAGACACAGGCATCCAGGAAAGTGTCACAGAGCAGGACAAGGACAGT
ACCTACAGCCTCAGCAGCACCCTGACGATGTCCAGTACTGAGTACCTAA
GTCATGAGTTGTACTCCTGTGAGATCACTCACAAGAGCCTGCCCTCCAC
CCTCATCAAGAGCTTCCAAAGGAGCGAGTGTCAGAGAGTGGACTAA.

SEQ ID NO: 7 is the amino acid sequence for the cPD-L1_3C8D3_dIgG2_heavy chain of the cPD-L1 3C chimeric antibody clone:

MGWSWIFLFLLSGTAGVLSEVQLQQSGPELVKPGASVKMSCKASGYTFT
DYVLHWVRQTNGKSLEWIGEINPSNGDTYYNQKFKGKATLTVDTSSSTA
YMQLTTLTSEDSAVYYCARSDYSNYVGFAYWGQGTLVTVSAASTTAPSV
FPLAPSCGSTSGSTVALACLVSGYFPEPVTVSWNSGSLTSGVHTFPSVL
QSSGLYSLSSMVTVPSSRWPSETFTCNVAHPASKTKVDKPVPKRENGRV
PRPPDCPKCPAPEMLGGPSVFIFPPKPKDTLLIARTPEVTCVVVDLDPE
DPEVQISWFVDGKQMQTAKTQPREEQFNGTYRVVSVLPIGHQDWLKGKQ
FTCKVNNKALPSPIERTISKARGQAHQPSVYVLPPSREELSKNTVSLTC
LIKDFFPPDIDVEWQSNGQQEPESKYRTTPPQLDEDGSYFLYSKLSVDK
SRWQRGDTFICAVMHEALHNHYTQKSLSHSPGK

wherein, Underline, signal sequence; Italic, FR; Bold, CDR; Bold and Underlined, CL; *, Stop; CDR1: GYTFTDYV (SEQ ID NO: 18); CDR2: INPSNGDT (SEQ ID NO: 19); and CDR3: CARSDYSNYVGFAYW (SEQ ID NO: 13).

SEQ ID NO: 8 is the nucleotide sequence for the cPD-L1_3C8D3_dIgG2_heavy chain of the cPD-L1 3C chimeric antibody clone (and encodies SEQ ID NO: 7):

ATGGGATGGAGCTGGATCTTTCTCTTTCTCTTGTCAGGAACTGCAGGTG
TCCTCTCTGAAGTCCAGCTGCAACAGTCTGGACCTGAGCTGGTGAAGCC
TGGGGCTTCAGTGAAGATGTCCTGCAAGGCTTCTGGATACACATTCACT
GACTATGTTTTGCACTGGGTGAGGCAGACCAATGGAAAGAGCCTTGAGT
GGATTGGAGAAATTAATCCTAGCAATGGTGATACTTACTACAACCAGAA
GTTCAAGGGCAAGGCCACATTGACTGTAGACACATCCTCCAGCACAGCC
TACATGCAGCTCACCACCCTGACATCTGAGGACTCTGCAGTCTATTACT
GTGCACGATCGGATTATAGTAACTACGTAGGATTTGCTTACTGGGGCCA
AGGGACTCTGGTCACTGTCTCTGCAGCTAGCACCACGGCCCCCTCGGTT
TTCCCACTGGCCCCCAGCTGCGGGTCCACTTCCGGCTCCACGGTGGCCC
TGGCCTGCCTGGTGTCAGGCTACTTCCCCGAGCCTGTAACTGTGTCCTG
GAATTCCGGCTCCTTGACCAGCGGTGTGCACACCTTCCCGTCCGTCCTG
CAGTCCTCAGGGCTCTACTCCCTCAGCAGCATGGTGACAGTGCCCTCCA
GCAGGTGGCCCAGCGAGACCTTCACCTGCAACGTGGCCCACCCGGCCAG
CAAAACTAAAGTAGACAAGCCAGTGCCCAAAAGAGAAAATGGAAGAGTT
CCTCGCCCACCTGATTGTCCCAAATGCCCAGCCCCTGAAATGCTGGGAG
GGCCTTCGGTCTTCATCTTTCCCCCGAAACCCAAGGACACCCTCTTGAT
TGCCCGAACACCTGAGGTCACATGTGTGGTGGTGGATCTGGACCCAGAA
GACCCTGAGGTGCAGATCAGCTGGTTCGTGGACGGTAAGCAGATGCAAA
CAGCCAAGACTCAGCCTCGTGAGGAGCAGTTCAATGGCACCTACCGTGT
GGTCAGTGTCCTCCCCATTGGGCACCAGGACTGGCTCAAGGGGAAGCAG
TTCACGTGCAAAGTCAACAACAAAGCCCTCCCATCCCCGATCGAGAGGA
CCATCTCCAAGGCCAGAGGGCAGGCCCATCAACCCAGTGTGTATGTCCT
GCCGCCATCCCGGGAGGAGTTGAGCAAGAACACAGTCAGCTTGACATGC
CTGATCAAAGACTTCTTCCCACCTGACATTGATGTGGAGTGGCAGAGCA
ATGGACAGCAGGAGCCTGAGAGCAAGTACCGCACGACCCCGCCCCAGCT
GGACGAGGACGGGTCCTACTTCCTGTACAGCAAGCTCTCTGTGGACAAG
AGCCGCTGGCAGCGGGGAGACACCTTCATATGTGCGGTGATGCATGAAG
CTCTACACAACCACTACACACAGAAATCCCTCTCCCATTCTCCGGGTAA
ATGA.

SEQ ID NO: 9 is the amino acid sequence for a CDR1: ESVEYYGTSL.

SEQ ID NO: 10 is the amino acid sequence for a CDR3: CQQSGKVPHTF.

SEQ ID NO: 11 is the amino acid sequence for a CDR3: ARGGGPDWYFDV.

SEQ ID NO: 12 is the amino acid sequence for a CDR3: CQQYSSYPLTF.

SEQ ID NO: 13 is the amino acid sequence for a CDR3: CARSDYSNYVGFAYW.

SEQ ID NO: 14 is a portion of the amino acid sequence for the cPD-L1 12C chi antibody clone: GDTFICAVMHEALHNHYTQK.

SEQ ID NO: 15 is the amino acid sequence for a CDR1: GFSLTSFG.

SEQ ID NO: 16 is the amino acid sequence for a CDR2: IWSGGST.

SEQ ID NO: 17 is the amino acid sequence for a CDR1: QDVGTA.

SEQ ID NO: 18 is the amino acid sequence for a CDR1: GYTFTDYV.

SEQ ID NO: 19 is the amino acid sequence for a CDR2: INPSNGDT.

SEQ ID NO: 20 is the nucleotide sequence for a pre-assembled guide RNA related to full length of canine CD274 cDNA (NM_001291972): CAGCAAATATCCTCATGTTTTGG.

SEQ ID NO: 21 is the nucleotide sequence of a forward primer of primer set 1 described herein: CCACTTGGTTCTACATGGCT.

SEQ ID NO: 22 is the nucleotide sequence of a reverse primer of primer set 1 described herein: CCTCAGCCTGACACATTAGTT.

SEQ ID NO: 23 is the nucleotide sequence of a forward primer of primer set 2 described herein: CCTGTCACCTCTGAACATGAA.

SEQ ID NO: 24 is the nucleotide sequence of a reverse primer of primer set 2 described herein: GGACTAAGCTCTAGGTTGTCC.

SEQ ID NO: 25 is the nucleotide sequence of a forward primer of primer set 3 described herein: GACTGGCTTTTAGGGCTTATGT.

SEQ ID NO: 26 is the nucleotide sequence of a reverse primer of primer set 3 described herein: ACACCCCACAAATTACTTCCATT.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the compositions, compounds and methods hereof may comprise many different configurations, forms, materials, and accessories.

Novel canine programmed death-ligand 1 (cPD-L1) antibodies and cPD-L1 antigen binding fragments are provided. The term “canine” includes all domestic dogs, Canis lupus familiaris or Canis familiaris, unless otherwise indicated. These antibodies and antigen binding fragments can be used as an immuno-oncology drug, for example. When administered, these antibodies and antigen binding fragments can increase tumor cell killing activity in a subject. In certain embodiments, the antibodies and antigen binding fragments hereof can inhibit the immunosuppressive function of cPD-L1 in the presence of cancer cells. Given that the expression of PD-L1 on T cells and natural killer (NK) cells has been reported, together with the blockade of PD-L1 on cancer cells by the antibodies and/or antigen binding fragments hereof, a direct effect of PD-L1 on T cells or other immune cells can also enhance the efficacy of the antibodies and antigen binding fragments hereof when administered to a subject, in addition to targeting the PD-L1 on cancer cells.

### Antibodies and Complementarity Determining Regions (CDRs)

Canine programmed cell death protein 1 (PD-1)/PD-L1 blockade antibodies are not widely available for dogs with invasive urothelial carcinoma (InvUC). Tumor regression in dogs with oral melanoma and soft tissue sarcomas has been reported in response to a canine chimeric monoclonal antibody targeting PD-L1. The antitumor effects of the antibody, however, were indeterminate as the role of concurrent medications in tumor regression was not known (Maekawa et al., A canine chimeric monoclonal antibody targeting PD-L1 and its clinical efficacy in canine oral malignant melanoma or undifferentiated sarcoma, Sci Rep 7:8951 (2017); Knapp et al., Phase I trial of piroxicam in 62 dogs bearing naturally occurring tumors, Cancer Chemother Pharmacol 29, 214-218 (1992); and Nemoto et al., Development and characterization of monoclonal antibodies against canine PD-1 and PD-L1, Vet Immunol Immunopathol 198:19-25 (2018)). In other work, anti-canine PD-1 and PD-L1 antibodies have been developed for diagnostic applications, but have not been tested therapeutically (Choi et al., Development of canine PD-1/PD-L1 specific monoclonal antibodies and amplification of canine T cell function, PLOS One 15: e0235518 (2020)). Accordingly, a canine immune checkpoint blockade antibody or anti-canine PD-L1 antibody is not conventionally available for translational research or treatment in dogs.

An immunotherapeutic PD-L1 antibody (or an antigen binding fragment thereof) is provided. As used herein, the term “antibody” refers to any form of immunoglobulin that exhibits the desired biological activity. Thus, it is used in the broadest sense and specifically covers, but is not limited to, monoclonal antibodies (including full length monoclonal antibodies), polyclonal antibodies, multi-specific antibodies (e.g., bispecific antibodies), caninized antibodies, fully canine antibodies, chimeric antibodies, and single domain antibodies. “Parental antibodies” are antibodies obtained by exposure of an immune system to an antigen prior to modification of the antibodies for an intended sue, such as caninization of an antibody for use as a canine therapeutic antibody.

The variable regions of each light/heavy chain pair form the antibody binding site. Thus, in general, an intact antibody has two binding sites. Except in bifunctional or bispecific antibodies, the two binding sites are, in general, identical. Typically, the variable domains of both the heavy and light chains comprise three hypervariable regions, also called CDRs, located between relatively conserved variable framework regions (FRs). The CDRs are usually flanked by the FRs, enabling binding to a specific epitope. In general, from N-terminal to C-terminal, both light and heavy chains variable domains comprise FR1, CDR1, FR2, CDR2, FR3, CDR3 and FR4.

As used herein, unless otherwise indicated, “antibody fragment” or “antibody binding fragment” refers to antigen binding fragments of antibodies, i.e. antibody fragments that retain the ability to bind specifically to the antigen bound by the full-length antibody. Examples of antigen binding fragments include, without limitation, Fab, Fab′, F(ab′) 2, and Fv fragments; diabodies; linear antibodies; single-chain antibody molecules (e.g., sc-Fv); nanobodies and multi-specific antibodies formed from antibody fragments. A “Fab fragment” is comprised of one light chain constant, one light chain variable, one CHI and one variable region-domains of one heavy chain. A “Fab fragment” can be the product of papain cleavage of an antibody. A “fragment crystallizable” (Fc) region is a tail region of an antibody that interacts with cell surface receptors and allows antibodies to activate the immune system. The Fc region contains at least two heavy chain fragments (i.e. two identical polypeptides) comprising the CH2 and CH3 domains of an antibody. The two heavy chain fragments can be held together by two or more disulfide bonds and by hydrophobic interactions of the CH3 domains.

A “Fab′ fragment” contains one light chain and a portion or fragment of one heavy chain that contains the heavy chain variable domain (VH) domain and the CH1 domain and also the region between the CH1 and CH2 domains, such that an interchain disulfide bond can be formed between the two heavy chains and two Fab′ fragments to form a F(ab′)₂ molecule.

In certain embodiments, the PD-L1 antibody (or an antigen binding fragment thereof) is a chimeric form of a caninized antibody (e.g., monoclonal) or an antigen binding fragment thereof that binds PD-L1 in a canine subject with specificity (e.g., has high binding affinity for PD-L1 in a canine subject). As used herein, a “chimeric antibody” is an antibody having the variable domain from a first antibody and the constant domain from a second antibody, where the first and second antibodies are from different species. For example, the variable segments of the genes (e.g., the framework and CDR portions) from, for example, a mouse antibody (e.g., a monoclonal or polyclonal antibody) can be used in conjunction with canine or human constant segments to produce the chimeric antibody. A typical therapeutic chimeric antibody is thus a hybrid protein composed of the variable or antigen-binding domain from a mouse antibody (e.g., one or more of the CDRs of a mouse antibody) and the constant or effector domain from a human or canine antibody, although other mammalian species can be used. A chimeric antibody can also include amino acid sequences obtained from a protein source other than an antibody.

In certain embodiments, the antibody or antigen binding fragment thereof binds canine PD-L1 with specificity. As used herein, an antibody or antigen binding fragment thereof binds “with specificity” to a polypeptide comprising a given antigen sequence (e.g., a portion of an amino acid sequence of a canine antigen, e.g., cPD-L1) if it binds to polypeptides comprising that portion of the amino acid sequence of the canine antigen, for example, cPD-L1, but does not bind to other canine proteins lacking that portion of the sequence of the canine antigen. For example, an antibody that specifically binds to a polypeptide comprising canine PD-L1 can bind to a FLAG-tagged form of canine PD-L1 but will not bind to other FLAG-tagged canine proteins with specificity. An antibody, or binding compound derived from the antigen-binding site of an antibody, binds to its canine antigen (or a variant or mutein thereof) “with specificity” when it has an affinity for that canine antigen or a variant or mutein thereof that is at least ten-times greater, at least fifteen-times greater, at least 20-times greater, or at least 100-times greater than its affinity for any other canine antigen tested.

The cPD-L1 antibody can comprise the amino acid sequence of SEQ ID NOS: 1 and/or 3, or SEQ ID NOS: 5 and/or 7. The cPD-L1 is encoded by the nucleotide sequence comprising SEQ ID NOS: 2 and/or 4, or SEQ ID NOS: 6 and/or 8.

In certain embodiments, the antibody (or an antigen binding fragment thereof) is caninized. In certain embodiments, the antibody (or an antigen binding fragment thereof) is completely caninized. In certain embodiments, the antibody (or an antigen binding fragment thereof) is incompletely caninized. As used herein, the term “caninized antibody” refers to forms of antibodies that contain sequences from both canine and non-canine (e.g., murine or rat) antibodies. In general, the caninized antibody can comprise substantially all of at least one, and typically two, variable domains, in which all or substantially all of the hypervariable loops correspond to those of a non-canine immunoglobulin (e.g., comprising 6 murine anti-canine PD-L1 CDRs as exemplified below), and all or substantially all of the parental framework. A caninized antibody can also refer to an antibody where point mutations are made changing one amino acid to reflect that commonly found at that position in canines versus that commonly found at that position in a mouse or rat species.

The caninized regions are those in which the amino acid identity at selected positions is changed from the original (i.e., rat or mouse) amino acid to ones that more closely reflect common canine amino acids at those position. These changes are made in an attempt to prevent or reduce host anti-antibody responses during or after therapy. These undesired anti-antibody responses are commonly found when the host (in this case, a dog) detects an unusual amino acid identity in a position of importance. As a non-limiting example, a dog can raise an undesired immune response to a lysine found at position 29 (Lys29) because dog antibodies rarely, if ever, have a lysine at position 29. The caninization process can then result in changing Lysine 29 to Alanine (Lys29Ala) in an attempt to avoid a strong immune response to the antibody which could have severe side effects. Most common caninization changes are placed in the variable domains of a therapeutic antibody, though it is possible to need caninization changes in one or more other antibody regions as well. The anti-PD-L1 antibody (or an antigen binding fragment thereof) can comprise a caninized murine PD-L1 antibody or an antigen binding fragment thereof.

In certain embodiments, the antibody or an antigen binding fragment thereof comprises a caninized antibody wherein all or a portion of the mouse (or other mammalian) CDR sequences are replaced with the corresponding canine CDR sequences or portion(s) thereof. The antibody or an antigen binding fragment thereof can comprise a fusion of one or more variable regions of mouse DNA with constant regions for canine DNA. In certain embodiments, caninized anti-canine PD-L1 antibodies are caninized mammalian (e.g., murine) anti-canine PD-L1 antibodies.

CDRs are those portions of variable regions of immunoglobulins (i.e., antibodies) and T cell receptors that participate in the binding of specific antigens, epitopes, or peptides. There are three CDRs (CDR1, CDR2, and CDR3), arranged non-consecutively, on an amino acid sequence of a variable domain of a complete antigen receptor, for example. As antigen receptors are typically comprised of two variable domains (on two different chains-heavy and light chain), there are six CDRs for each antigen receptor that can collectively come into contact with an antigen.

The antibodies (or antigen binding fragments thereof) can comprise one or more genetically modified CDRs. In certain embodiments, a CDR (e.g., of the antibody or antigen binding fragment thereof) comprises AAS, SEQ ID NO: 9, and/or SEQ ID NO: 10, or has at least 80%, or about 80%-100% (such as about 80% to 100%, 80% to about 100%, or 80-100%), sequence identity to AAS, SEQ ID NO: 9, and/or SEQ ID NO: 10. In certain embodiments a CDR comprises at least 80%, or between about 85%-95% (such as about 85% to 95%, 85% to about 95%, or 85-95%), sequence identity to AAS, SEQ ID NO: 9, and/or SEQ ID NO: 10. In certain embodiments, a CDR comprises about 87%-93% (such as about 87% to 93%, 87% to about 93%, or 87-93%) sequence identity to AAS, SEQ ID NO: 9, and/or SEQ ID NO: 10. In certain embodiments a CDR comprises about 90% (such as 90%) sequence identity to AAS, SEQ ID NO: 9, and/or SEQ ID NO: 10. The ranges specified in this paragraph are inclusive of the stated end points and all 1% increments therein. In certain embodiments, the CDR (e.g., of the antibody or antigen binding fragment thereof) comprises AAS, SEQ ID NO: 9, and/or SEQ ID NO: 10 or a sequence that is 90%, 95%, 98%, or 99% identical to the sequence of AAS, SEQ ID NO: 9, and/or SEQ ID NO: 10. In certain embodiments, the CDR comprises a sequence that is substantially identical to AAS, SEQ ID NO: 9, and/or SEQ ID NO: 10.

In certain embodiments, a CDR comprises SEQ ID NO: 17, WTS, and/or SEQ ID NO: 12, or has at least about 80%, or about 80%-100% (such as about 80% to 100%, 80% to about 100%, or 80-100%), sequence identity (or is substantially identical) to SEQ ID NO: 17, WTS, and/or SEQ ID NO: 12. In certain embodiments a CDR comprises between about 85%-95% (such as about 85% to 95%, 85% to about 95%, or 85-95%) sequence identity to SEQ ID NO: 17, WTS, and/or SEQ ID NO: 12. In certain embodiments, a CDR comprises between about 87%-93% (such as about 87% to 93%, 87% to about 93%, or 87-93%) sequence identity to SEQ ID NO: 17, WTS, and/or SEQ ID NO: 12. In certain embodiments a CDR comprises about 90% (such as 90%) sequence identity to SEQ ID NO: 17, WTS, and/or SEQ ID NO: 12. The ranges specified in this paragraph are inclusive of the stated end points and all 1% increments therein. In certain embodiments, the CDR (e.g., of the antibody or antigen binding fragment thereof) comprises SEQ ID NO: 17, WTS, and/or SEQ ID NO: 12 or a sequence that is 90%, 95%, 98%, or 99% identical to the sequence of SEQ ID NO: 17, WTS, and/or SEQ ID NO: 12.

In certain embodiments, a CDR comprises SEQ ID NO: 15, SEQ ID NO: 16, and/or SEQ ID NO: 11 or has at least about 80%, or has about 80%-100% (such as about 80% to 100%, 80% to about 100%, or 80-100%), sequence identity (or is substantially identical) to SEQ ID NO: 15, SEQ ID NO: 16, and/or SEQ ID NO: 11. In certain embodiments a CDR comprises between about 85%-95% (such as about 85% to 95%, 85% to about 95%, or 85-95%) sequence identity to SEQ ID NO: 15, SEQ ID NO: 16, and/or SEQ ID NO: 11. In certain embodiments, a CDR comprises between about 87%-93% (such as about 87% to 93%, 87% to about 93%, or 87-93%) sequence identity to SEQ ID NO: 15, SEQ ID NO: 16, and/or SEQ ID NO: 11. In certain embodiments a CDR comprises about 90% (such as 90%) sequence identity to SEQ ID NO: 15, SEQ ID NO: 16, and/or SEQ ID NO: 11. The ranges specified in this paragraph are inclusive of the stated end points and all 1% increments therein. In certain embodiments, the CDR (e.g., of the antibody or antigen binding fragment thereof) comprises SEQ ID NO: 15, SEQ ID NO: 16, and/or SEQ ID NO: 11 or a sequence that is 90%, 95%, 98%, or 99% identical to the sequence of SEQ ID NO: 15, SEQ ID NO: 16, and/or SEQ ID NO: 11.

In certain embodiments, a CDR comprises SEQ ID NO: 18, SEQ ID NO: 19, and/or SEQ ID NO: 13 or has at least about 80%, or has about 80%-100% (such as about 80% to 100%, 80% to about 100%, or 80-100%), sequence identity (or is substantially identical) to SEQ ID NO: 18, SEQ ID NO: 19, and/or SEQ ID NO: 13. In certain embodiments a CDR comprises between about 85%-95% (such as about 85% to 95%, 85% to about 95%, or 85-95%) sequence identity to SEQ ID NO: 18, SEQ ID NO: 19, and/or SEQ ID NO: 13. In certain embodiments, a CDR comprises between about 87%-93% (such as about 87% to 93%, 87% to about 93%, or 87-93%) sequence identity to SEQ ID NO: 18, SEQ ID NO: 19, and/or SEQ ID NO: 13. In certain embodiments a CDR comprises about 90% (such as 90%) sequence identity to SEQ ID NO: 18, SEQ ID NO: 19, and/or SEQ ID NO: 13. The ranges specified in this paragraph are inclusive of the stated end points and all 1% increments therein. In certain embodiments, the CDR (e.g., of the antibody or antigen binding fragment thereof) comprises SEQ ID NO: 18, SEQ ID NO: 19, and/or SEQ ID NO: 13 or a sequence that is 90%, 95%, 98%, or 99% identical to the sequence of SEQ ID NO: 18, SEQ ID NO: 19, and/or SEQ ID NO: 13.

In certain embodiments, an anti-cPD-L1 antibody or antigen binding fragment, can comprise one or more CDRs independently comprising at least about 80% sequence identity to AAS, SEQ ID NO: 9, and/or SEQ ID NO: 10. In certain embodiments, an anti-cPD-L1 antibody or antigen binding fragment can comprise one or more CDRs independently comprising at least about 80% sequence identity to SEQ ID NO: 17, WTS, and/or SEQ ID NO: 12. In certain embodiments, a cPD-L1 antibody or antigen binding fragment can comprise one or more CDRs independently comprising at least about 80% sequence identity to SEQ ID NO: 15, SEQ ID NO: 16, and/or SEQ ID NO: 11. In certain embodiments, a cPD-L1 antibody or antigen binding fragment can comprise one or more CDRs independently comprising at least about 80% sequence identity to SEQ ID NO: 18, SEQ ID NO: 19, and/or SEQ ID NO: 13.

In certain embodiments, an antibody hereof (or an antigen binding fragment thereof) comprises two or more of the CDRs described herein.

In certain embodiments, the antibody hereof is a monoclonal antibody. In certain embodiments, the monoclonal antibody is a murine antibody. In certain embodiments, the monoclonal antibody is a caninized antibody. In certain embodiments, the monoclonal antibody hereof is a caninized murine antibody. The antibodies (and/or their antigen binding fragments) can be isolated antibodies (or isolated antigen binding fragments). “Isolated antibody” or “isolated antigen binding fragment” refers to the purification status and in such context means the molecule is substantially free of other biological molecules such as nucleic acids, proteins, lipids, carbohydrates, or other material such as cellular debris and growth media. Generally, unless otherwise specified, the term “isolated” is not intended to refer to a complete absence of such material or to an absence of water, buffers, or salts, unless they are present in amounts that substantially interfere with experimental or therapeutic use of the binding compound as described herein.

In certain embodiments, the antibody is a recombinant antibody or antigen binding fragment. In certain embodiments, the heavy chain variable domain and the light chain variable domain are connected by a flexible linker to form a single chain antibody.

The antibody or antigen binding fragment can be a Fab fragment. The antibody or antigen binding fragment can be a Fab′ fragment. The antibody or antigen binding fragment can be a F(ab′)₂ molecule. In certain embodiments, the antibody or antigen binding fragment is a diabody. In certain embodiments, the antibody or antigen binding fragment is a domain antibody.

Nucleic acids (including isolated nucleic acids) encoding any of the antibodies, fragments, and/or portions thereof (including CDRs) are also provided. In certain embodiments, nucleic acids that encode any of the light chains or caninized antibodies or portions thereof are provided. Similarly, nucleic acids that encode any of the heavy chains or caninized antibodies or portions thereof are provided.

The nucleic acid sequence encoding an antibody hereof can comprise SEQ ID NO: 2. In certain embodiments, the nucleic acid sequence encoding an antibody hereof comprises at least about 80% sequence identity to SEQ ID NO: 2, or has about 80%-100% (such as about 80% to 100%, 80% to about 100%, or 80-100%) sequence identity (or is substantially identical), to SEQ ID NO: 2. In certain embodiments, the nucleic acid sequence encoding an antibody hereof comprises between about 85%-95% (such as about 85% to 95%, 85% to about 95%, or 85-95%) sequence identity to SEQ ID NO: 2. In certain embodiments, the nucleic acid sequence encoding an antibody hereof comprises 87%-93% (such as about 87% to 93%, 87% to about 93%, or 87-93%) sequence identity to SEQ ID NO: 2. the nucleic acid sequence encoding an antibody hereof comprises about 90% (such as 90%) sequence identity to SEQ ID NO: 2. The ranges specified in this paragraph are inclusive of the stated end points and all 1% increments therein. In certain embodiments, the nucleic acid sequence encoding an antibody hereof comprises SEQ ID NO: 2 or a sequence that is 90%, 95%, 98%, or 99% identical to the sequence of SEQ ID NO: 2.

The nucleic acid sequence encoding an antibody hereof can comprise SEQ ID NO: 4. In certain embodiments, the nucleic acid sequence encoding an antibody hereof comprises at least about 80% sequence identity to SEQ ID NO: 4, or has about 80%-100% (such as about 80% to 100%, 80% to about 100%, or 80-100%) sequence identity (or is substantially identical) to SEQ ID NO: 4. In certain embodiments, the nucleic acid sequence encoding an antibody hereof comprises between about 85%-95% (such as about 85% to 95%, 85% to about 95%, or 85-95%) sequence identity to SEQ ID NO: 4. In certain embodiments, the nucleic acid sequence encoding an antibody hereof comprises 87%-93% (such as about 87% to 93%, 87% to about 93%, or 87-93%) sequence identity to SEQ ID NO: 4. The nucleic acid sequence encoding an antibody hereof comprises about 90% (such as 90%) sequence identity to SEQ ID NO: 4. The ranges specified in this paragraph are inclusive of the stated end points and all 1% increments therein. In certain embodiments, the nucleic acid sequence encoding an antibody hereof comprises SEQ ID NO: 4 or a sequence that is 90%, 95%, 98%, or 99% identical to the sequence of SEQ ID NO: 4.

In certain embodiments, the nucleic acid sequence encoding an antibody hereof comprises SEQ ID NOS: 2 and 4.

In certain embodiments, the nucleic acid sequence encoding an antibody hereof comprises SEQ ID NO: 6, has at least 80% sequence identity to, or has about 80%-100% (such as about 80% to 100%, 80% to about 100%, or 80-100%) sequence identity (or is substantially identical) to, SEQ ID NO: 6. In certain embodiments, the nucleic acid sequence encoding an antibody hereof comprises between about 85%-95% (such as about 85% to 95%, 85% to about 95%, or 85-95%) sequence identity to SEQ ID NO: 6. In certain embodiments, the nucleic acid sequence encoding an antibody hereof comprises 87%-93% (such as about 87% to 93%, 87% to about 93%, or 87-93%) sequence identity to SEQ ID NO: 6. the nucleic acid sequence encoding an antibody hereof comprises about 90% (such as 90%) sequence identity to SEQ ID NO: 6. The ranges specified in this paragraph are inclusive of the stated end points and all 1% increments therein. In certain embodiments, the nucleic acid sequence encoding an antibody hereof comprises SEQ ID NO: 6 or a sequence that is 90%, 95%, 98%, or 99% identical to the sequence of SEQ ID NO: 6.

In certain embodiments, the nucleic acid sequence encoding an antibody hereof comprises SEQ ID NO: 8, has at least 80$ sequence identity to, or has about 80%-100% (such as about 80% to 100%, 80% to about 100%, or 80-100%) sequence identity (or is substantially identical) to, SEQ ID NO: 8. In certain embodiments, the nucleic acid sequence encoding an antibody hereof comprises between about 85%-95% (such as about 85% to 95%, 85% to about 95%, or 85-95%) sequence identity to SEQ ID NO: 8. In certain embodiments, the nucleic acid sequence encoding an antibody hereof comprises 87%-93% (such as about 87% to 93%, 87% to about 93%, or 87-93%) sequence identity to SEQ ID NO: 8. the nucleic acid sequence encoding an antibody hereof comprises about 90% (such as 90%) sequence identity to SEQ ID NO: 8. The ranges specified in this paragraph are inclusive of the stated end points and all 1% increments therein. In certain embodiments, the nucleic acid sequence encoding an antibody hereof comprises SEQ ID NO: 8 or a sequence that is 90%, 95%, 98%, or 99% identical to the sequence of SEQ ID NO: 8.

In certain embodiments, the nucleic acid sequence encoding an antibody hereof comprises SEQ ID NOS: 6 and 8.

In certain embodiments, the antibodies (or antigen binding fragments thereof) to canine PD-L1 comprise one or more of the CDRs described herein and/or bind to an amino acid sequence of PD-L1. In certain embodiments, the dissociation constant (K_{D}) for caninized antibody-canine PD-L1 binding is between about 4.0 nmol/L to 10.0 nmol/L (such as 4.0 nmol/L to about 10.0 nmol/L, about 4.0 nmol/L to about 10.0 nmol/L, or about 4.0 nmol/L to 10.0 nmol/L). In certain embodiments, the antibodies (e.g., caninized antibodies) or their antigen-binding fragments bind to canine PD-L1 with a K_{D} of at or between about 4.2 nmol/L to 9.8 nmol/L (such as 4.2 nmol/L to about 9.8 nmol/L, about 4.2 nmol/L to about 9.8 nmol/L, or about 4.2 nmol/L to 9.8 nmol/L). In certain embodiments, the antibodies (e.g., caninized antibodies) or their antigen-binding fragments bind to canine PD-L1 with a K_{D} of at or between about 4.4 nmol/L to 9.6 nmol/L (such as 4.4 nmol/L to about 9.6 nmol/L, about 4.4 nmol/L to about 9.6 nmol/L, or about 4.4 nmol/L to 9.6 nmol/L). In certain embodiments, the antibodies (e.g., caninized antibodies) or their antigen-binding fragments bind to canine PD-L1 with a K_{D} of at or between about 4.6 nmol/L to 9.4 nmol/L (such as 4.6 nmol/L to about 9.4 nmol/L, about 4.6 nmol/L to about 9.4 nmol/L, or about 4.6 nmol/L to 9.4 nmol/L). In certain embodiments, the antibodies (e.g., caninized antibodies) or their antigen-binding fragments bind to canine PD-L1 with a K_{D} of at or between about 4.8 nmol/L to 9.2 nmol/L (such as 4.8 nmol/L to about 9.2 nmol/L, about 4.8 nmol/L to about 9.2 nmol/L, or about 4.8 nmol/L to 9.2 nmol/L). In certain embodiments, the antibodies (e.g., caninized antibodies) or their antigen-binding fragments bind to canine PD-L1 with a K_{D} of at or between about 5.0 nmol/L to 9.0 nmol/L (such as 5.0 nmol/L to about 9.0 nmol/L, about 5.0 nmol/L to about 9.0 nmol/L, or about 5.0 nmol/L to 9.0 nmol/L). In certain embodiments, the antibodies (e.g., caninized antibodies) or their antigen-binding fragments bind to canine PD-L1 with a K_{D} of at or between about 5.2 nmol/L to 8.8 nmol/L (such as 5.2 nmol/L to about 8.8 nmol/L, about 5.2 nmol/L to about 8.8 nmol/L, or about 5.2 nmol/L to 8.8 nmol/L). In certain embodiments, the antibodies (e.g., caninized antibodies) or their antigen-binding fragments bind to canine PD-L1 with a K_{D} of at or between about 5.4 nmol/L to 8.6 nmol/L (such as 5.4 nmol/L to about 8.6 nmol/L, about 5.4 nmol/L to about 8.6 nmol/L, or about 5.4 nmol/L to 8.6 nmol/L). In certain embodiments, the antibodies (e.g., caninized antibodies) or their antigen-binding fragments bind to canine PD-L1 with a K_{D} 8.6 nmol/L. In certain embodiments, the antibodies (e.g., caninized antibodies) or their antigen-binding fragments bind to canine PD-L1 with a K_{D} of at or between about 5.6 nmol/L to 8.4 nmol/L (such as 5.6 nmol/L to about 8.4 nmol/L, about 5.6 nmol/L to about 8.4 nmol/L, or about 5.6 nmol/L to 8.4 nmol/L). In certain embodiments, the antibodies (e.g., caninized antibodies) or their antigen-binding fragments bind to canine PD-L1 with a K_{D} of at or between about 5.8 nmol/L to 8.2 nmol/L (such as 5.8 nmol/L to about 8.2 nmol/L, about 5.8 nmol/L to about 8.2 nmol/L, or about 5.8 nmol/L to 8.2 nmol/L). In certain embodiments, the antibodies (e.g., caninized antibodies) or their antigen-binding fragments bind to canine PD-L1 with a K_{D} of at or between about 6.0 nmol/L to 8.0 nmol/L (such as 6.0 nmol/L to about 8.0 nmol/L, about 6.0 nmol/L to about 8.0 nmol/L, or about 6.0 nmol/L to 8.0 nmol/L). In certain embodiments, the antibodies (e.g., caninized antibodies) or their antigen-binding fragments bind to canine PD-L1 with a K_{D} of at or between about 6.2 nmol/L to 7.8 nmol/L (such as 6.2 nmol/L to about 7.8 nmol/L, about 6.2 nmol/L to about 7.8 nmol/L, or about 6.2 nmol/L to 7.8 nmol/L). In certain embodiments, the antibodies (e.g., caninized antibodies) or their antigen-binding fragments bind to canine PD-L1 with a K_{D} of at or between about 6.4 nmol/L to 7.6 nmol/L (such as 6.4 nmol/L to about 7.6 nmol/L, about 6.4 nmol/L to about 7.6 nmol/L, or about 6.4 nmol/L to 7.6 nmol/L). In certain embodiments, the antibodies (e.g., caninized antibodies) or their antigen-binding fragments bind to canine PD-L1 with a K_{D} of at or between about 6.6 nmol/L to 7.4 nmol/L (such as 6.6 nmol/L to about 7.4 nmol/L, about 6.6 nmol/L to about 7.4 nmol/L, or about 6.6 nmol/L to 7.4 nmol/L). The ranges specified in this paragraph are inclusive of the stated end points and all 0.1 increments encompassed thereby.

The antibodies or their antigen-binding fragments can stimulate antigen-specific memory responses to a tumor or pathogen. In certain embodiments, the antibodies or their antigen-binding fragments can stimulate an antibody response in vivo in a subject. In certain embodiments, the antibodies or their antigen-binding fragments can stimulate an immune response in an animal subject (e.g., a canine animal subject). The term “immune response” refers to the action of, for example, lymphocytes, antigen-presenting cells, phagocytic cells, granulocytes, and soluble macromolecules produced by the above cells or the liver (including antibodies, cytokines, and complement) that can result in selective damage to, destruction, or elimination of the mammalian body (e.g., canine body) results from cancer cells, cells, or tissues infected with invading pathogens or pathogens.

In certain embodiments, the antibodies or their antigen binding fragments can bind to canine PD-L1 and also block the binding of canine PD-L1 to PD-1. In certain embodiments, the caninized antibodies and their antigen-binding fragments hereof can bind to canine PD-L1 and block the binding of canine PD-L1 to PD-1.

The antibodies or antigen-binding fragments can be used for the preparation of a medicament for treating cancer in a canine subject. The antibodies (or antigen binding fragments thereof) can be administered in unit dosage forms and/or compositions containing one or more pharmaceutically acceptable carriers, adjuvants, diluents, excipients, and/or vehicles, and combinations thereof. The term “administering” and its formatives generally refer to any and all means of introducing compounds described herein to the host subject including, but not limited to, by oral, intravenous, intramuscular, subcutaneous, transdermal, inhalation, buccal, ocular, sublingual, vaginal, rectal, and like routes of administration.

Alternatively, or in conjunction, the use of any of the antibodies or antibody fragments hereof is provided for diagnostic use. In certain embodiments, an expression vector is provided comprising an isolated nucleic acid encoding any of the caninized murine anti-canine PD-L1 antibodies or antigen-binding fragments hereof. Host cells are also provided that comprise one or more expression vectors described herein. In particular embodiments, these nucleic acids, expression vectors, or polypeptides can be useful in methods for preparing an antibody.

Pharmaceutical compositions are also provided. The pharmaceutical compositions can comprise one or more of the antibodies, antigen binding fragments described herein, antigenic peptides (including isolated antigenic peptides) of canine PD-L1, fusion proteins comprising the canine PD-L1 antigenic peptides, nucleic acids encoding the antigenic fragments and/or fusion proteins hereof, the expression vectors comprising such nucleic acids, or any combination thereof, and a pharmaceutically acceptable carrier or diluent. The term “composition” generally refers to any product comprising more than one ingredient, including without limitation the antibodies or antigen binding fragments thereof. In certain embodiments, the pharmaceutical composition comprises the caninized antibody, a chimerized antibody, or an antigen binding fragment thereof and a pharmaceutically acceptable excipient.

The compositions can be prepared from isolated antibodies or antigen binding fragments thereof or from salts, solutions, hydrates, solvates, and other forms of the antibodies or antigen binding fragments thereof. The compositions can be prepared from various amorphous, non-amorphous, partially crystalline, crystalline, and/or other morphological forms of the antibodies or antigen binding fragments thereof, and the compositions can be prepared from various hydrates and/or solvates of the compounds.

The antibodies or antigen binding fragments thereof can be formulated as pharmaceutical compositions and administered to a mammalian host, such as a human or canine subject, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intra-sternal, intracranial, intra-tumoral, intramuscular, topical, inhalation and/or subcutaneous routes. Indeed, in at least one embodiment, a compound and/or composition can be administered directly into the blood stream, into muscle, or into an internal organ.

For example, in at least one embodiment, the antibodies or antigen binding fragments thereof can be systemically administered (orally, for example) in combination with a pharmaceutically acceptable vehicle, such as an inert diluent or an assimilable edible carrier. For oral therapeutic administration, the antibody or an antigen binding fragment thereof can be combined with one or more excipients and used in the form of ingestible tablets, buccal tablets, troches, capsules, elixirs, suspensions, syrups, wafers, and the like. The percentage of the compositions and preparations can vary and can be between about 1 to about 99% weight of the active ingredient(s) and a binder, excipients, a disintegrating agent, a lubricant, and/or a sweetening agent (as are known in the art). The amount of antibody or an antigen binding fragment thereof in such therapeutically useful compositions is such that an effective dosage level will be obtained.

The preparation of parenteral compounds/compositions under sterile conditions, for example, by lyophilization, can readily be accomplished using standard pharmaceutical techniques well-known to those skilled in the art. In at least one embodiment, the solubility of a compound used in the preparation of a parenteral composition can be increased by the use of appropriate formulation techniques, such as the incorporation of solubility-enhancing agents.

As previously noted, the antibodies or antigen binding fragments thereof can also be administered via infusion or injection (e.g., using needle (including microneedle) injectors and/or needle-free injectors). Solutions of the active composition can be aqueous, optionally mixed with a nontoxic surfactant and/or can contain carriers or excipients such as salts, carbohydrates and buffering agents (preferably at a pH of from 3 to 9), but, for some applications, they can be more suitably formulated as a sterile non-aqueous solution or as a dried form to be used in conjunction with a suitable vehicle such as sterile, pyrogen-free water or phosphate-buffered saline (PBS). For example, dispersions can be prepared in glycerol, liquid PEGs, triacetin, and mixtures thereof and in oils. Under ordinary conditions of storage and use, these preparations can further contain a preservative to prevent the growth of microorganisms.

### Methods for Treatment

A method of treating animal cancers is provided using the antibodies or antigen binding fragments hereof, particularly invasive urothelial carcinoma (InvUC) in canines, which comprises greater than about 90% of dog bladder cancers. In at least one embodiment, the method comprises administering a therapeutically effective amount of any of the antibodies or antigen binding fragments thereof to a canine subject (e.g., to treat a cancer). The cancer can be, for example, any canine cancer including, without limitation, InvUC. In certain embodiments, administration of the therapeutically effective amount of the caninized antibody, chimeric antibody, or an antigen binding fragment thereof inhibits PD-L1/PD-1 interaction in the subject. In certain embodiments, administration of the therapeutically effective amount of the caninized antibody, chimeric antibody, or an antigen binding fragment thereof increases the activity of an immune cell in the subject.

The subject can be a mammal. The subject can be a canine. The subject can be a companion dog.

“Effective amount” or “therapeutically effective amount” refers to an amount of a therapeutic agent (e.g., an antibody or antigen binding fragment thereof), or composition comprising the same, that elicits the desired biological or medicinal response in a subject (i.e., a tissue, organ, or organism, such as a vertebrate, e.g., mammal, such as a human or a canine) that is being sought by a researcher, veterinarian, medical doctor, or other clinician, which includes, but is not limited to, imaging and/or alleviation of the signs and/or symptoms of the disease or disorder being treated. In one aspect, the effective amount is an amount of an active agent which can treat or alleviate the signs and/or symptoms of the disease at a reasonable benefit/risk ratio applicable to any medical treatment. An “effective amount” or “therapeutically effective amount” with respect to use in treatment refers to an amount of the active agent/antibody in a preparation which, when administered as part of a desired dosage regimen (e.g., to a mammal, such as a human or canine) alleviates a symptom, ameliorates a condition, or slows the onset of disease according to clinically acceptable standards for the disorder or condition to be treated or the cosmetic purpose, e.g., at a reasonable benefit/risk ratio applicable to any medical treatment.

Combined with the teachings provided herein, by choosing among the various active agents and weighing factors such as potency, relative bioavailability, subject body weight, severity of adverse side-effects and mode of administration, an effective prophylactic or therapeutic treatment regimen can be planned which does not cause substantial unwanted toxicity and yet is effective to treat the particular subject.

Depending upon the type of cancer, the route of administration and/or whether the antibodies or antigen binding fragments thereof are administered locally or systemically, a wide range of permissible dosages are contemplated herein. The amount of the composition required for use in treatment (e.g., the therapeutically or prophylactically effective amount or dose) will vary not only with the particular application and dosage structure, but also with the salt selected (if applicable) and the characteristics of the subject (such as, for example, species, breed, age, condition, sex, the subject's body surface area and/or mass, tolerance to drugs) and will ultimately be at the discretion of the attendant physician, veterinarian, clinician, or otherwise.

Therapeutically effective amounts or doses can range, for example, from about 0.05 mg/kg of subject body weight to about 30.0 mg/kg of subject body weight, or from about 0.01 mg/kg of subject body weight to about 5.0 mg/kg of subject body weight, including but not limited to 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of subject body weight. The total therapeutically effective amount of the compound can be administered in single or divided doses and can, at the practitioner's discretion, fall outside of the typical range given herein.

Higher doses can be required for parenteral administration. The effective amount for any particular application can vary depending on such factors as the disease or condition being treated, the particular antibodies being administered, the size of the subject, or the severity of the disease or condition. One of ordinary skill in the art can empirically determine the effective amount of a particular antibody, antigen binding fragment thereof, and/or other therapeutic agent without necessitating undue experimentation. A maximum dose can be used, that is, the highest safe dose according to some medical judgment. Multiple doses per day can be used to achieve appropriate systemic levels of compounds. Appropriate systemic levels can be determined by, for example, measurement of the patient's peak or sustained plasma level of the drug. “Dose” and “dosage” are used interchangeably herein.

Adjusting the dose to achieve maximal efficacy based on the methods described and other methods well-known in the art is well within the capabilities of the ordinarily skilled artisan. Dosage can be adjusted appropriately to achieve desired drug levels, local or systemic, depending upon the mode of administration. For example, the dose for intravenous administration can vary from one order to several orders of magnitude lower per day. If the response in a subject is insufficient at such doses, even higher doses (or effective higher doses by a different, more localized delivery route) can be employed to the extent that subject tolerance permits. Multiple doses per day are contemplated to achieve appropriate systemic levels of the compound.

Any effective regimen for administering the antibodies or antigen binding fragments thereof can be used. The dosages can be single or divided and can be administered according to a wide variety of protocols, including q.d., b.i.d., t.i.d., or even every other day, biweekly (b.i.w.), once a week, once a month, once a quarter, and the like. In each of these cases it is understood that the effective amounts described herein correspond to the instance of administration, or alternatively to the total daily, weekly, month, or quarterly dose, as determined by the dosing protocol.

For example, an antibody can be administered as a single dose, or the dose can be divided and administered as a multiple-dose daily regimen. Further, a staggered regimen, for example, one to five days per week can be used as an alternative to daily treatment. Such intermittent or staggered daily regimen is considered equivalent to daily treatment. The subject can be treated with multiple injections of an antibody or antigen binding fragment thereof to treat cancer. The subject can be injected multiple times (e.g., approximately 2-50×) with an antibody or an antigen binding fragment thereof, for example, at 12-72 hours intervals or at 48-72 hours intervals. Additional injections of an antibody or an antigen binding fragment thereof can be administered to the subject at an interval of days or months after the initial injection(s), and the additional injections can prevent the recurrence of the cancer.

The methods can be used in combination with one or more additional therapies and/or active agents. Such additional therapies include, without limitation, additional immunotherapies, the administration of a DNA damage response pathway inhibitor, chemotherapy, radiation and/or a surgery.

### Methods for Predicting and Modeling Anti-Cancer Activity of a Test Compound

Companion dogs naturally develop several types of cancer that in many respects resemble clinical cancer in human patients. Although mouse models are the most commonly used animal model in cancer research, they do not possess collective features such as tumor heterogeneity, mutational landscape, cancer molecular subtypes, and immune cell responsiveness that mimics the conditions desired for optimal animal models. Studies in the mouse models can be complemented by other models such as specific forms of naturally occurring cancer in pet dogs. This can be especially beneficial when developing or studying new immune-oncology drugs such as novel immune checkpoint inhibitors (ICIs) or their combination regimens where companion dogs can offer naturally occurring cancer in the context of an intact immune system and an aggressive heterogeneous cancer. The development of canine ICIs can expand comparative oncology approaches to improve the current therapeutic efficacy of immunotherapies in human cancer. Accordingly, the canine studies of immune-oncology drugs can be convertible into knowledge that informs and prioritizes new immune-oncology therapy in humans. Conventionally, however, the challenge has been that ICIs that target canine immune checkpoint molecules such as cPD-1 and cPD-L1 are not available.

In the development of immuno-oncology drugs, particularly immunotherapeutic antibodies, translation of the discoveries from mouse models to clinical trials has been hindered by many biological differences between mice and humans, such as no cross-reactivity between species and/or that the mouse physiological system can react differently to cancer and other pathologic insults than does the human or dog system. For example, if anti-human or canine PD-L1 antibody does not recognize mouse PD-L1 protein, the human or canine antibody's therapeutic efficacy cannot be evaluated in a syngeneic mouse model. Human immune system-engrafted mice have been developed for translational research to overcome this constraint. Indeed, pembrolizumab, an anti-human PD-1 antibody, showed tumor growth inhibition and CD8+ T cell activation in the humanized NSG (HuNSG) mice that received tumor implant from patient-derived xenografts (PDX) (see, e.g., Wang et al., Humanized mice in studying efficacy and mechanisms of PD-1-targeted cancer immunotherapy, FASEB J 32:1537-1549 (2018)). Despite human tumor-bearing human immune system engrafted mice being an important model for preclinical immuno-oncology research, there are considerable obstacles such as a limited source of human cells and tissues, immune rejection, and high cost in these humanized mouse models (see, e.g., Yong et al., Humanized mice as unique tools for human-specific studies, Arch Immunol Ther Exp (Warsz) 66:245-266 (2018)).

As an alternative mouse model for immunotherapeutic antibody development, mice that are humanized for immune checkpoint molecules are commercially available. For example, the humanized PD-L1 mouse has been generated by replacing the mouse PD-L1 gene (cd274) with the human PD-L1 gene (CD274) using the clustered regularly interspaced short palindromic repeats (CRISPR)/CAS9 strategy. The humanized PD-L1 mouse can be used for evaluating anti-human PD-L1 antibody's therapeutic efficacy in vivo; however, the canine PD-L1 gene knock-in mouse model hereof that can be used for evaluating anti-canine PD-L1 antibody's therapeutic efficacy has heretofore not been available, which has caused a major roadblock for developing canine immune checkpoint inhibitors such as PD-1/PD-L1 blockade antibodies. The PD-L1 mouse model hereof can be used not only for preclinical immune-oncology research, but also as a translational research tool to bridge the gap between canines and humans and, thus, increase success rates of immunotherapy applications in humans.

Methods for predicting and modeling (e.g., optimizing) anti-cancer activity of a test compound in a secondary subject using the antibodies or antigen binding fragments hereof are also provided. In certain embodiments, the antibodies or antigen binding fragments thereof are caninized and evaluated in canines (e.g., the primary subject), and those test compounds with the highest success are advanced to human trials (e.g., wherein the secondary subject is human).

In certain embodiments, a method for predicting and modeling anti-cancer activity of a test compound in a subject comprises generating a population of mice that express canine PD-L1 on a cell surface; evaluating toxicity risk and/or efficacy in treating a cancer of a set of test compounds in the population of PD-L1 mice; and selecting one or more test compounds of the set that satisfy established toxicity risk and/or efficacy criteria. The set of test compounds can comprise, for example, at least a compound that inhibits (or is reasonably expected could inhibit) PD-L1/PD-1 interaction in the subject.

In certain embodiments, the method further comprises evaluating oral bioavailability, adsorption, distribution, metabolism, and excretion (ADME) values of the set of test compounds in the population of mice that contain canine PD-L1 and PD-1. In certain embodiments, the method further comprises evaluating oral bioavailability and ADME values of the selected one or more test compounds in the population of PD-L1 mice.

The method can further comprise evaluating the toxicity risk and/or efficacy of the one or more selected compounds in treating a cancer in a human or canine cohort.

The toxicity risk and/or efficacy criteria can be established pursuant to known protocols.

Generating the population of PD-L1 mice can further comprise replacing a mouse cd274 gene in a population of mice with a canine PD-L1 gene using CRISPR or other known technologies.

The canine models hereof can be useful in studies of InvUC-specific therapies. This concept is supported by the data that human InvUC can replicate in canines and the similarities between human InvUC and canine InvUC. Canine InvUC mimics human InvUC in presentation, pathology, local invasion, distant metastases (lung, etc., in greater than 50% of cases), and chemotherapy response (see, e.g., Cekanova et al., Molecular imaging of cyclooxygenase-2 in canine transitional cell carcinomas in vitro and in vivo, Cancer Prev Res (Phila) 6:466-76 (2013); Fulkerson et al., Naturally occurring canine invasive urinary bladder cancer: A complementary animal model to improve the success rate in human clinical trials of new cancer drugs, Int J Genomics, 6589529 (2017); Knapp et al., Cisplatin versus cisplatin combined with piroxicam in a canine model of human invasive urinary bladder cancer, Cancer Chemother Pharmacol 46:221-226 (2000); Knapp et al., Urinary bladder cancer in dogs, a naturally occurring model for cancer biology and drug development, ILAR J 55:100-118 (2014); Lin et al., Targeting canine bladder transitional cell carcinoma with a human bladder cancer-specific ligand, Mol Cancer 10:9 (2011); Patrick et al., Classification of canine urinary bladder urothelial tumours based on the World Health Organization/International Society of Urological Pathology consensus classification, J Comp Pathol 135:190-199 (2006); Sommer et al., Naturally-occurring canine invasive urothelial carcinoma: A model for emerging therapies, Bladder Cancer 4:149-159 (2018); and Suarez-Bonnet et al., Expression of cell cycle regulators, 14-3-3sigma and p53 proteins, and vimentin in canine transitional cell carcinoma of the urinary bladder, Urol Oncol 33:332 e1-7 (2015)). With InvUC comprising two percent (2%) of the roughly six million new canine cancer cases each year in the United States, ample number of dogs are available for translational studies (see, e.g., Davis and Ostrander, Domestic dogs and cancer research: a breed-based genomics approach, ILAR J 55:59-68 (2014)). Canine clinical trials in which the test subjects continue life as pets following the studies can be win-win with benefits to each dog and the knowledge gained to help people and pet dogs. Accordingly, dogs offer an excellent opportunity to advance PD-1/PD-L1 blockade therapies in humans. Successful treatment approaches in rodents can then be evaluated in dogs, and those with the highest success moved into human trials.

### EXAMPLES

The present disclosure will be better understood by reference to the following Examples, which are provided as exemplary examples and not by way of limitation.

### Materials

BT549 human breast cancer and MB49 mouse bladder cancer cell lines were obtained from the American Type Culture Collection (ATCC) (Manassas, VA) and Sigma-Aldrich (St. Louis, MO), respectively. Canine bladder cancer cell line, K9TCC, was generated in the Knapp Lab. (see Igase et al. (2020), supra). Human embryonic kidney cell line HEK293FT was obtained from Thermo Fisher Scientific (Waltham, MA). Cell lines were validated by short tandem repeat DNA fingerprinting using the AmpFISTR Identifier PCT Amplification Kit (Thermo Fisher Scientific, Waltham, MA) according to manufacturer's instructions.

The cells were tested for Mycoplasma using the Mycoplasma PCR Detection Kit (ABM).

Cells were grown in Dulbecco's Modified Eagle Medium (DMEM) or DMEM/Nutrient Mixture F-12 (DMEM/F-12) medium supplemented with 10% fetal bovine serum (FBS) for no more than 15 passages.

For stable expression of PD-L1, cDNA of canine PD-L1 (Sino Biological, Wayne, PA) was inserted into a pGIPZ vector (Horizen Discovery, Waterbeach, UK) as described in Lim et al. (2016), supra. Using a pGIPZ-shPD-L1/Flag-cPD-L1 dual-expression construct to knockdown endogenous human PD-L1 and reconstitute Flag-cPD-L1 simultaneously, endogenous PD-L1 knockdown and Flag-cPD-L1 expressing BT549 cell lines were established. See, e.g., Lim et al., EGFR signaling enhances aerobic glycolysis in triple negative breast cancer cells to promote tumor growth and immune escape, Cancer Res (2016).

Lentivirus was packaged by co-transfecting transfer plasmids with plasmid pMD2.G (Addgene #12259) and pCMV dR8.2 (Addgene #12263) to HEK293FT cells with X-tremeGENE HP (Roche Diagnostics, Indianapolis, IN), and the supernatant was harvested for lentiviral transduction. Selection with 1 μg/mL puromycin (InvivoGen, San Diego, CA) was routinely performed to maintain ectopic gene expression.

For mouse PD-L1 knockout, mouse PD-L1 double nickase plasmid (Santa Cruz Biotechnology, Dallas, TX) was transfected into MB49 cells using X-tremeGENE transfection reagent.

For canine PD-L1 overexpression MB49 cells (MB49cPDL1), mouse PD-L1 KO MB49 cells were infected with lentivirus carrying pGIPZ-Flag-cPD-L1 followed by selection with puromycin.

All animal work was approved by the Purdue Animal Care and Use Committee (PACUC) at Purdue University.

For flow cytometry analysis, MB49 or BT549 cells were washed twice with ice-cold cell staining buffer (Biolegend, San Diego, CA) and stained with cIgG control or 12C10E4 cIgG for 1 hour at 4° C. After three washes with staining buffer, cell samples were stained with Alexa Fluor 488-conjugated anti-canine IgG specific secondary antibody for 30 minutes at 4° C. Cell samples were loaded on BD LSRFortessa (BD, Franklin Lakes, NJ) for analysis. Data analysis was performed on FlowJo v9 software (BD). Every hour, green fluorescent signal was measured and quantified by IncuCyte S3 (Sartorius, Goettingen, Germany). The Image analysis was performed according to the manufacturer's protocol.

### Example 1

### Creation and Selection of Anti-Canine PD-L1 Monoclonal Antibodies

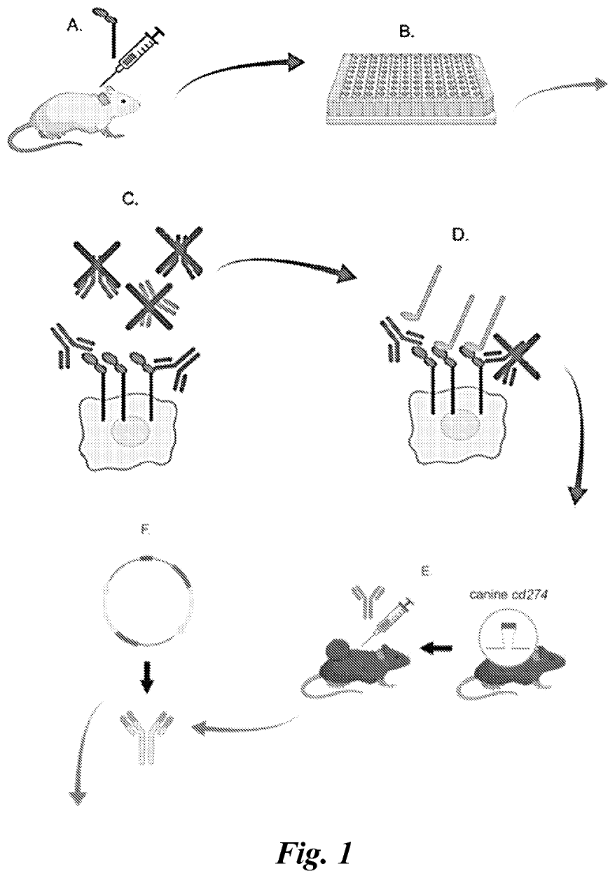

Monoclonal antibodies 3C8D3 (mAb 3C) and 12C10E4 (mAb 12C) were generated via conventional hybridoma procedures using A/J mice immunized with the extracellular domain of canine programmed death-ligand 1 (cPD-L1) (attached to a human Fc tag) at the Vanderbilt University Antibody and Protein Resource Core Facility. Briefly, splenocytes were isolated from the immunized mice and fused with SP2/0 myeloma cells (see FIG. 1). Supernatants from isolated clones were screened for the ability to block the canine programmed cell death protein 1 (cPD-1)/cPD-L1 interaction through cPD-L1 expressing cell-based enzyme-linked immunosorbent assays (ELISAs), with the mAbs 3C and 12C selected for further study. Clonal antibodies were purified from supernatants and the same assays were repeated. Anti-cPD-L1 mAbs were successfully generated using these conventional hybridoma procedures.

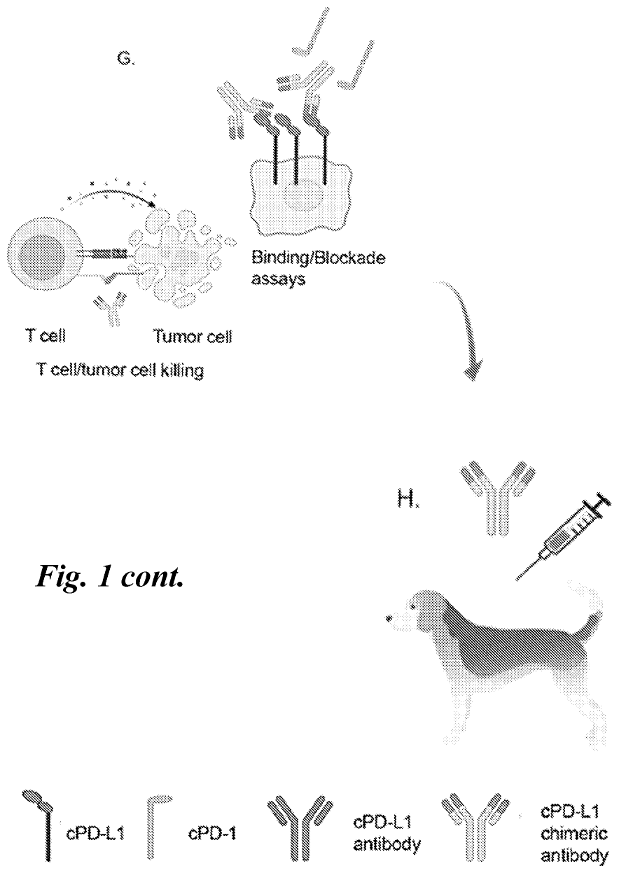

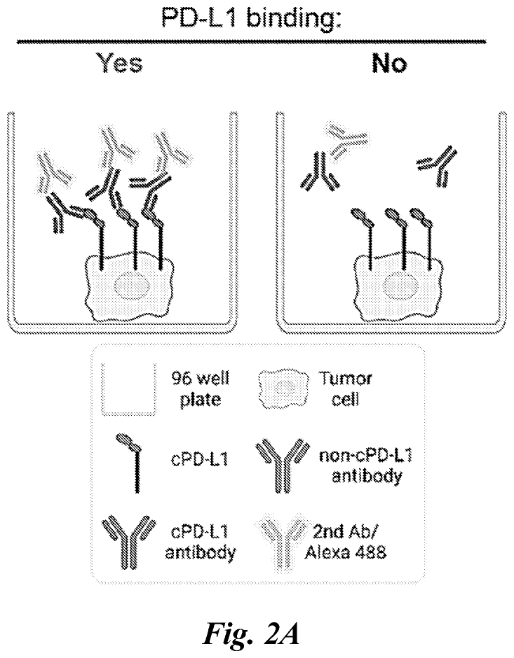

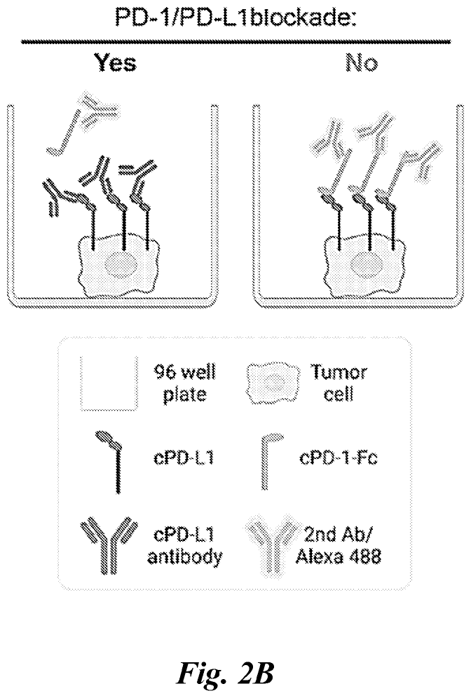

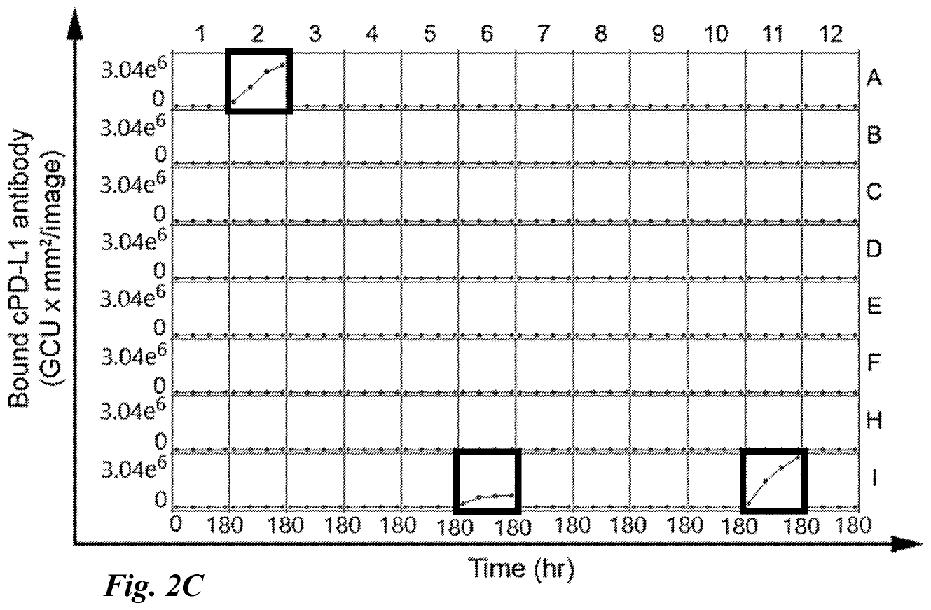

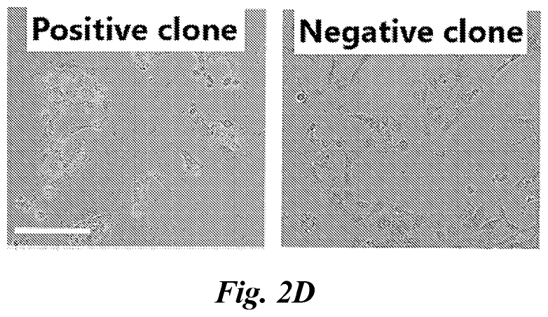

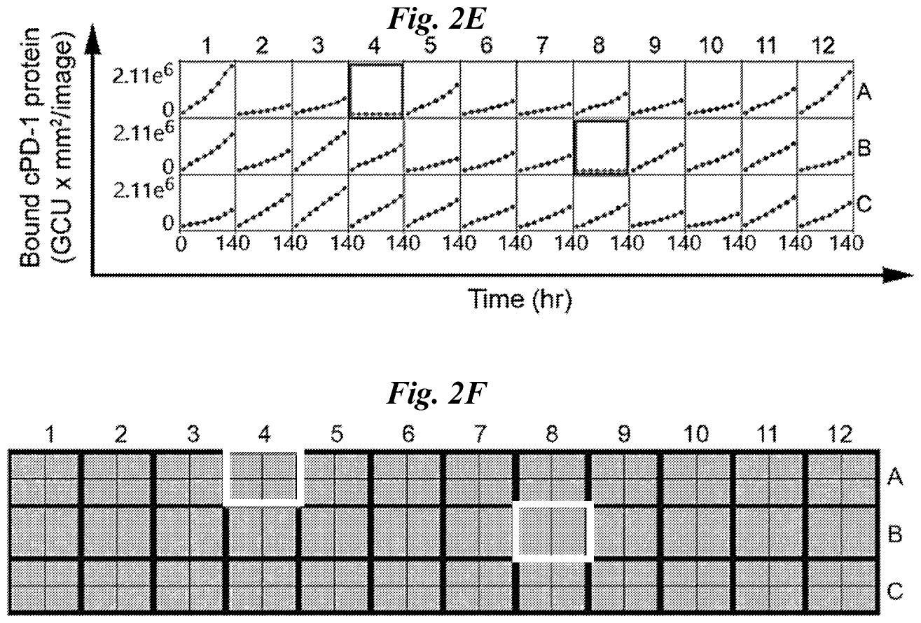

To screen for antibodies that could block the cPD-1/cPD-L1 interaction for use in a therapeutic setting, live cell-based antibody binding and PD-1/PD-L1 blockade assays were developed (see FIGS. 2A and 2B) similar to those previously published. See, e.g., Li et al., Eradication of triple-negative breast cancer cells by targeting glycosylated PD-L1, Cancer Cell 33:187-201 e10 (2018); Lim et al., Deubiquitination and stabilization of PD-L1 by SCN5, Cancer Cell (2016); Li et al., Glycosylation and stabilization of programmed death ligand-1 suppresses T-cell activity, Nat Commun 7:12632 (2016).

Human cancer cell line BT549 cells expressing cPD-L1 were seeded on 96-well or 384-well plates. cPD-L1 antibodies (from hybridomas) and Alexa Fluor® 488-conjugated anti-mouse IgG fc specific secondary antibody were added, and green fluorescence signal was measured to quantify the amount of bound PD-L1 antibody by the IncuCyte® S3 Live-Cell Analysis System (Sartorius AG, Gottingen, Germany) (FIG. 2A).

BT549 cells expressing cPD-L1 were seeded on 96-well or 384-well plates. cPD-1-human Fc (hFc) protein, Alexa Fluor® 488-conjugated anti-human IgG Fc specific secondary antibody and/or cPD-L1 antibody were added and then green fluorescence signal was measured to quantify the amount of bound PD-1 protein by IncuCyte® S3 Live-Cell Analysis System (Sartorius AG, Gottingen, Germany) (FIG. 2B).

Among over 2,000 hybridomas, 154 clones were screened against membrane-localized cPD-L1 protein by the live cell-based antibody binding assay. Clonal antibodies were purified from supernatants and the same assays were rerun. FIG. 2C shows a kinetic graph of the quantitative binding of PD-L1 antibodies on BT549 cells expressing cPD-L1 at every 3-hour time point. The positive clones are highlighted in bolded boxes. FIG. 2D shows representative images (taken at 18 hours) of cPD-L1 antibody binding, with the green-fluorescent merged images of cPD-L1 expressing cells shown.

Ten of these clones had the ability to block cPD-L1/cPD-1 interaction. FIG. 2E shows a kinetic graph of the quantitative binding of PD-1 protein on BT549 cells expressing cPD-L1 at every 3-hour time points after the addition of cPD-L1 antibodies. The positive clones that blocked the interaction of PD-L1/PD-1 proteins are highlighted in black boxes. FIG. 2F shows representative images (taken at 18 hours) of the cPD-L1 blockade, with the green-fluorescent merged images of cPD-L1 expressing cells shown. Note the lack of fluorescence (in the squares highlighted by white boxes) due to the antibody binding to PD-L1 and blocking the interaction with cPD-1.

Representative positive clones are shown in FIGS. 2B-2F. Based on the specificity, binding affinity, and PD-1/PD-L1 blockade efficacy, mAb 3C and mAb 12C were selected for further analysis.

### Example 2

### Generation of the Canine PD-L1 Knock-In Mouse as a Pre-Clinical Model

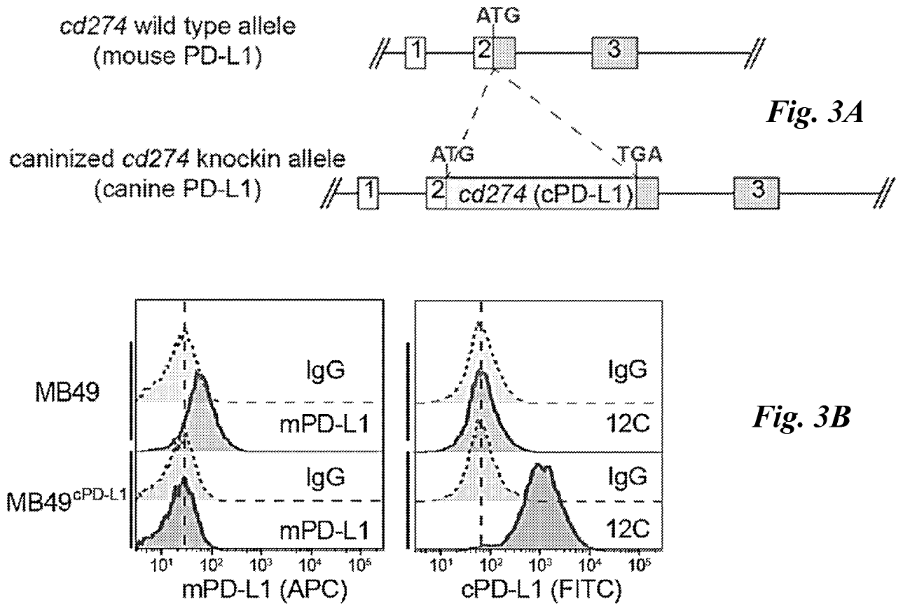

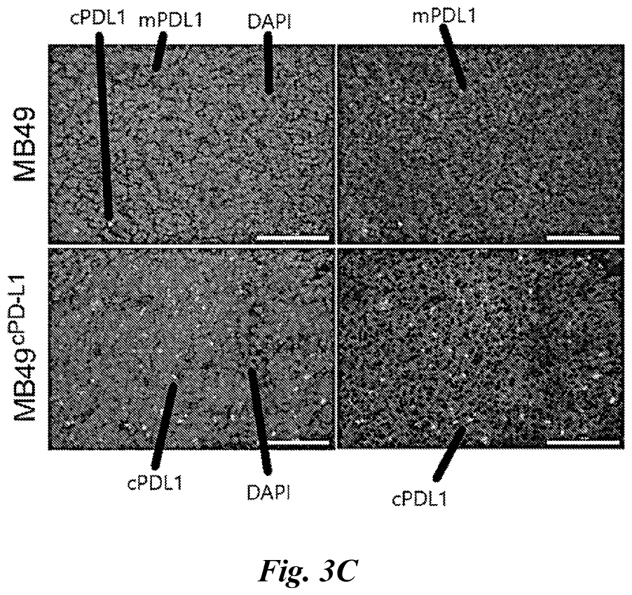

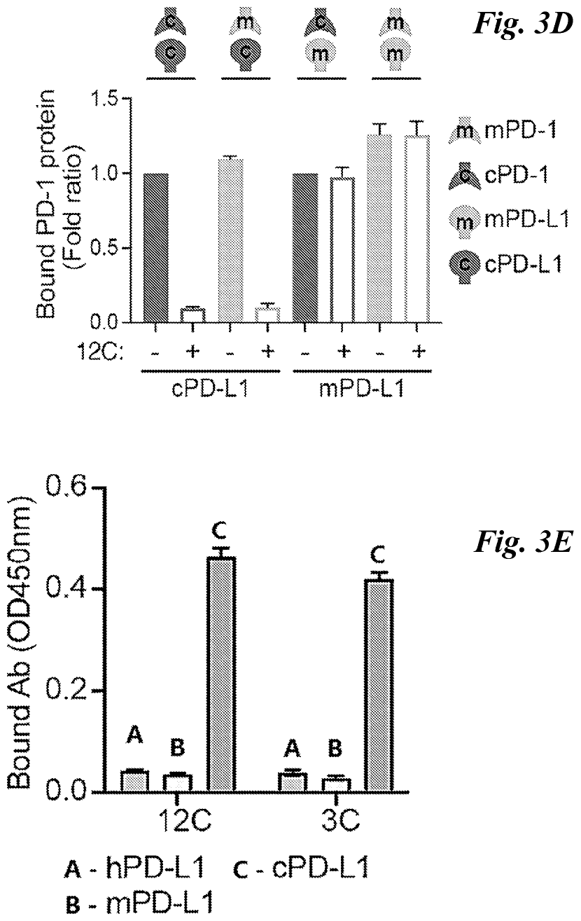

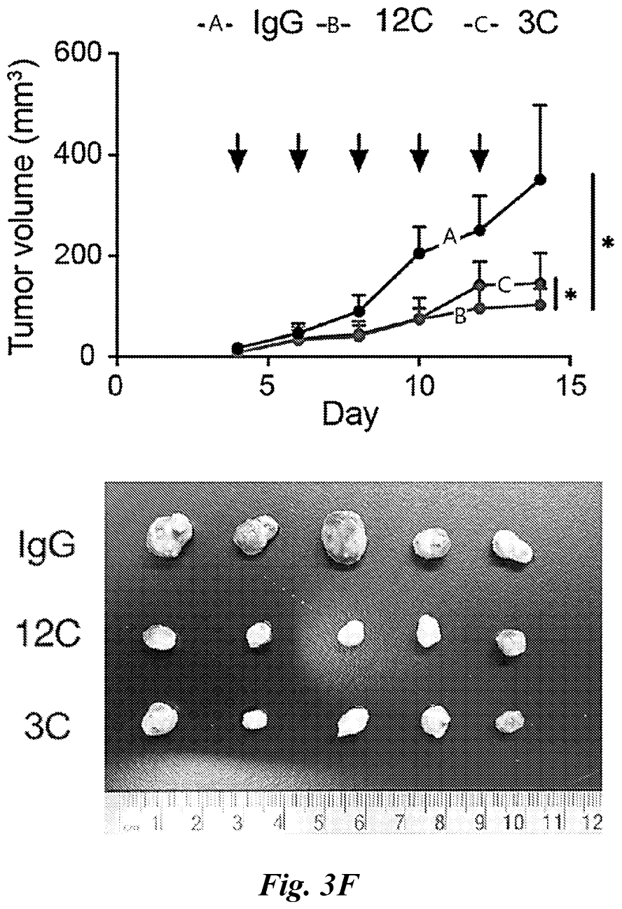

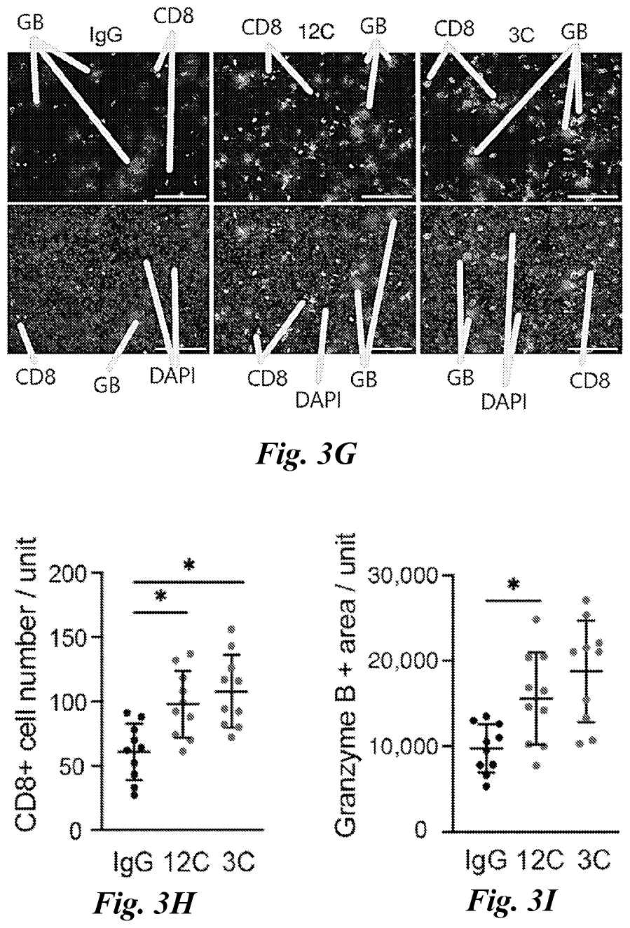

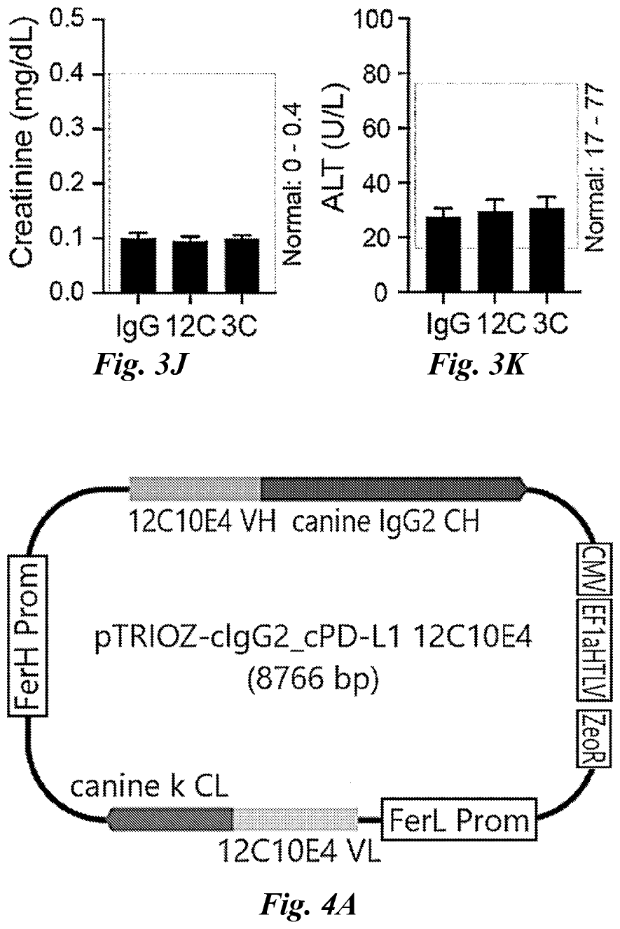

To further assess the clinical use of the cPD-L1 antibody as an immunotherapeutic drug, its therapeutic efficacy was evaluated in an in vivo model. To do this, (C57BL/6 background) mice were generated that expressed a canine form of PD-L1. This was accomplished by generating cPD-L1 mice in which the mouse cd274 (PD-L1) gene was replaced with cPD-L1 using the CRISPR knock-in mouse strategy using a long single-strand DNA (ssDNA) donor and CRISPR ribonucleoproteins (FIG. 3A).

More specifically, the Efficient Additions with ssDNA inserts-CRISPR (Easi-CRISPR) method for human CD247 knock-in mouse generation was employed to create mice that expressed PD-L1 on the cell surface (C57BL/6 background) by replacing the mouse CD274 with canine CD274. See Quadros et al., Easi-CRISPR: a robust method for one-step generation of mice carrying conditional and insertion alleles using long single-stranded DNA (ssDNA) donors and CRISPR ribonucleoproteins, Genome Biol 18:92 (2017). Easi-CRISPR is a targeting strategy in which long ssDNA donors are injected with preassembled crRNA+tracrRNA+Cas9 ribonucleoprotein (ctRNP) complexes into mouse zygotes to generate targeted insertion alleles in the resulting live offspring (here replacing the mouse cd274 (PD-L1) gene with the canine PD-L1 gene). The long ssDNA (a full length of canine CD274 cDNA; NM_001291972) was injected with pre-assembled guide RNA (gRNA, CAGCAAATATCCTCATGTTTTGG (SEQ ID NO: 20)) and Cas9 ribonucleoprotein (ctRNP) complexes into mouse zygotes. The ssDNA and sgRNA were synthesized at Integrated DNA Technologies (IDT, Coralville, IA, USA). All animal experiments for the knock-in mouse generation performed were approved by the Purdue Animal Care and Use Committee (PACUC) at Purdue University (West Lafayette, IN). C57BL/6N female mice at 4 weeks of age (Envigo, Indianapolis, IN, USA) were superovulated and then mouse zygotes were obtained by mating C57BL/6N males with the superovulated females.

Pronuclei of one-cell stage fertilized mouse embryos were injected with 20 ng/μl Cas9 protein, 10 ng/μl sgRNA, and 5 ng/μl ssDNA. Microinjections and mouse transgenesis were performed as described (40). Mouse genomic DNA was extracted from the tail tip and then used for the genotyping (Primer set 1 forward, 5′-CCACTTGGTTCTACATGGCT-3′ (SEQ ID NO: 21 Primer set 1 reverse, 5′-CCTCAGCCTGACACATTAGTT-3′ (SEQ ID NO: 22); Primer set 2 forward, 5′-CCTGTCACCTCTGAACATGAA-3′ (SEQ ID NO: 23); Primer set 2 reverse, 5′-GACTAAGCTCTAGGTTGTCC-3′ (SEQ ID NO: 24); Primer set 3 forward, 5′-GACTGGCTTTTAGGGCTTATGT-3′ (SEQ ID NO: 25); Primer set 3 reverse, 5′-ACACCCCACAAATTACTTCCATT-3′ (SEQ ID NO: 26)) and sequencing (Primer set 3 forward, 5′-GACTGGCTTTTAGGGCTTATGT-3′ (SEQ ID NO: 25); Primer set 3 reverse, 5′-ACACCCCACAAATTACTTCCATT-3′ (SEQ ID NO: 26)) to verify the location of insertion and DNA sequence of canine CD274.

To evaluate the therapeutic efficacy of the cPD-L1 antibody in a syngeneic animal model, a mouse bladder cancer MB49 cell line was generated that expressed cPD-L1 (MB49^{cPD-L1}) by knocking out mPD-L1 and re-expressing cPD-L1 (FIGS. 3B and 3C). FIG. 3B shows a flow cytometric analysis of membrane located cPD-L1 protein in MB49 cells expressing cPD-L1 (MB49^{cPD-L1}).

### Example 3

### Interaction of cPD-1 or mPD-1 Protein with cPD-L1 or mPD-L1 In Vitro with or without cPD-L1 Antibody (mAb 12C) Treatment

Although MB49^{cPD-L1} and the canine PD-L1 mice expressed cPD-L1 protein instead of mPD-L1 protein, the canine PD-L1 mice expressed mouse PD-1 protein. Accordingly, it was first examined if the cPD-L1 protein interacts with mPD-1 protein before evaluating the therapeutic efficacy of cPD-L1 antibody in the canine PD-L1 mice.

To measure immune receptor and ligand interaction, His-tagged canine or mouse PD-L1 protein (cPD-L1-His or mPD-L1-His, respectively) were incubated on a nickel-nitriloacetic acid (Ni-NTA) coated 96-well plates. The plates were then incubated with recombinant Fc-tagged protein for 1 hour, and the secondary antibodies used were anti-human or canine IgG Fc-specific Alexa 488 dye conjugate (Jackson ImmunoResearch Inc., West Grove, PA). Fluorescence intensity of Alexa fluor 488 dye was measured by microplate reader (Synergy Neo2; BioTek Instruments, Inc., Winooski, VT).

The binding of cPD-L1 and mPD-1 was similar to the cognate cPD-L1 and cPD-1 pair (OD450 was measured to quantify the amount of bonded PD-1 protein (FIG. 3D)). Consistently, the cPD-L1 antibody (mAb 12C) efficiently blocked cPD-L1/mPD-1 interaction as well as cPD-L1/cPD-1 but did not block mPD-L1/mPD-1 or mPD-L1/cPD-1 interactions (as the present cPD-L1 antibodies did not recognize mPD-L1) (FIGS. 3D and 3E).

### Example 4

### Evaluation of Therapeutic Efficacy of cPD-L1 Antibodies in Canine PD-L1 Mice

All procedures with the canine PD-L1 B mice (C57BL/c background strain; 6- to 8-week-old) were conducted under guidelines approved by the PACUC at Purdue University. Mice were divided according to the mean tumor volume in each group. MB49^{cPD-L1} (2×10⁵ cells in 25 μL of medium mixed with 25 μL of Matrigel Basement Membrane Matrix (BD Biosciences, San Jose, CA)) were injected into the flank of the caninized PD-L1 mice.

Following the establishment of MB49^{cPD-L1} tumors in the cPD-L1 mice, the mice were divided into a control group, a mAb 12C treatment group, and a mAb 3C treatment group, with mice within each subset divided according to mean tumor volume in each group.

For treatment with antibodies, 100 μg of cPD-L1 antibody (mAb 12C or mAb 3C clone) or control mouse IgG (BioXCell) was injected intraperitoneally on days 4, 6, 8, 10, and 12 after tumor cell inoculation when tumor size was approximately 30 to 40 mm³. Tumors were measured every other day with a caliper, and tumor volume was calculated using the following formula:

$$π / 6 \times length \times {width}^{2} .$$

Tumors were dissected at the endpoint (n=8 per group) (FIG. 3F).

Immunofluorescence staining was performed of the protein expression pattern of CD8 and granzyme B in the MB49 tumor masses from the IgG-treated mice (control group), 12C-treated mice, and the 3C-treated mice (FIGS. 3G-3I). Tumor masses were frozen in an optimal cutting temperature (OTC) block immediately after excision. Cryostat sections of 5 μm thickness were attached to saline-coated slides. Cryostat sections were fixed with 4% paraformaldehyde for 30 minutes at room temperature and blocked with blocking solution (1% bovine serum albumin, 2% donkey and/or chicken serum, and 0.1M phosphate buffered saline (PBS)) at room temperature for 30 minutes. Samples were stained with primary antibodies against CD8 and granzyme B overnight at 4° C., followed by secondary antibodies at room temperature for 1 hour. Nuclear staining was performed with Hoechst 33342 (Thermo Fisher Scientific, Waltham, MA). The stained sections were visualized by automated microscopy (Lionheart LX; BioTek Instruments, Inc., Winooski, VT). Granzyme B positive area and the number of CD8 positive cytotoxic T lymphocytes (CTLs) were assessed per high power field (200×). Fourteen randomly chosen microscope fields from 4 serial sections in each tissue block were examined for the number of CD8 positive CTL and granzyme B positive areas for each tissue.

CD8 (FIG. 3H) and granzyme B (FIG. 3I) were also quantified using BioTek Gen5 Data Analysis Software (Agilent Technologies, Santa Clara, CA) (n=10). The treatment with either mAb 12C or mAb 3C significantly reduced the tumor size (FIG. 3F) and increased the number of infiltrating cytotoxic T cells relative to mice treated with control IgG as measured by CD8+ and granzyme B expression (FIGS. 3G-3I).

Additionally, the effect of treatment on the PD-L1 mice was assessed at the conclusion of treatment by measuring mice kidney (FIG. 3J) and liver (FIG. 3K) functions. The data support that both cPD-L1 antibodies enhanced anti-tumor immunity in the PD-L1 syngeneic mouse model and demonstrated good safety profiles as the mice maintained body weight, and there was no changes to kidney function as assed by serum creatinine or liver enzyme activity. The in vitro and in vivo validation results indicated that the cPD-L1 antibodies that recognized canine PD-L1 effectively inhibited the PD-1/PD-L1 pathway and enhanced mouse anti-tumor immunity.

### Example 5

### Characterization of the Caninized PD-L1 Chimeric Antibody

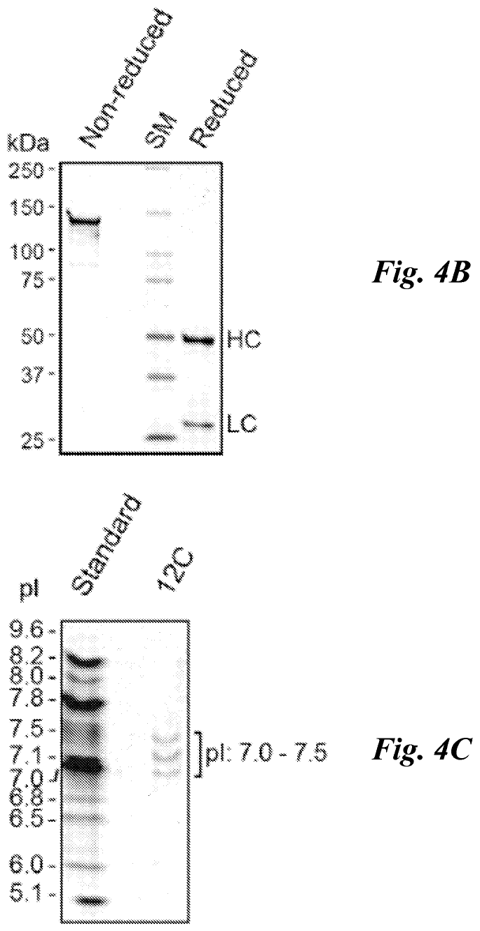

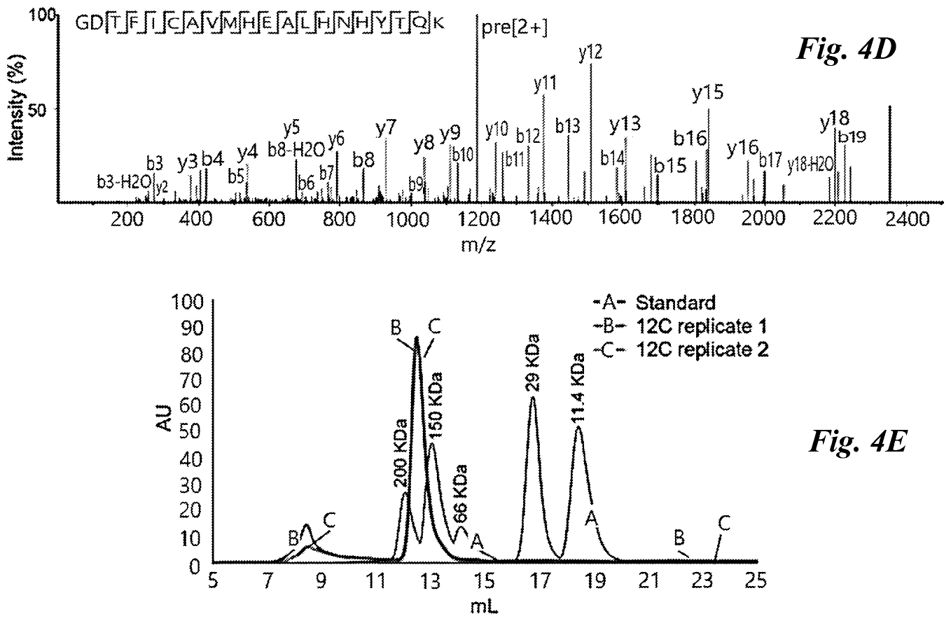

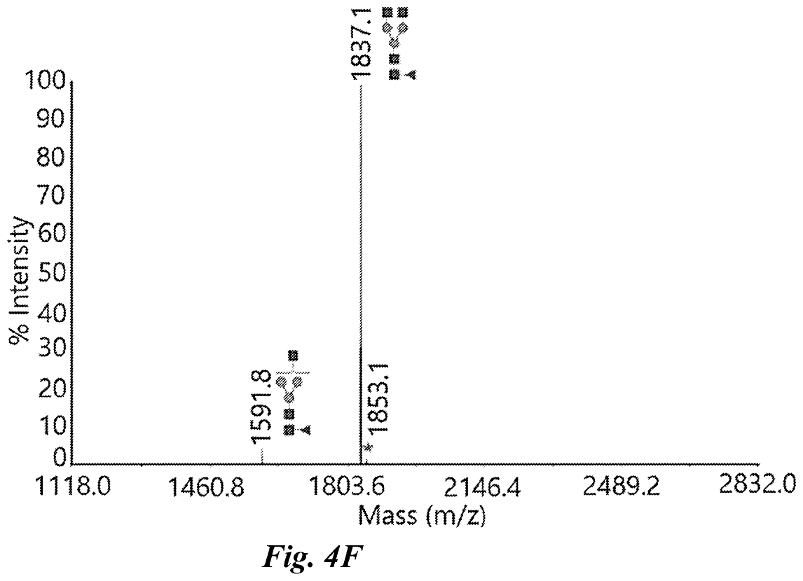

For the clinical use of the canine PD-L1 antibodies in dogs, mAb 12C was caninized by replacing the mouse constant domain with canine IgG2 (equivalent to human IgG1) constant domains. Briefly, full-length variable heavy (VH) and variable light (VL) RNA transcripts obtained from hybridoma clones were sequenced by 5′/3′ rapid amplification of cDNA ends (RACE) and codon-optimized for CHO VL and VH chains were cloned into the pTRIOZ-cIgGB-ck vector (InvivoGen, San Diego, CA, USA), a vector designed for high-yield production of whole monoclonal antibodies with the use of a single plasmid. Then the constant light and heavy chains were replaced with canine kappa light constant chain and canine IgG2 heavy constant chain: pTRIOZ-cIgG2-12C10E4. FIG. 4A shows a schematic representation of a cPD-L1 (mAb 12C) chimeric antibody expression construct (pTRIOZ-cIgG2-cPD-L1 mAb 12C). The chimeric cPD-L1 antibody retained the cPD-L1 binding VH and VL chains of the mouse hybridoma.

Plasmids encoding the 12C chimeric antibody, pTRIOZ cIgG2 12C10E4, were transfected into ExpiCHO-S cells following the transfection kit instructions (GIBCO, A29133). ExpiCHO-S cells were cultured with ExpiCHO Expression Medium (Thermo Fisher Scientific, Waltham, MA) in a shaker incubator set at 120 rpm, 37° C. and 8.0% CO₂. Cells were collected 10 days post-transfection at 4,000×g and 4° C. for 20 minutes. The antibody supernatant passed through a 0.22-μm filter and neutralized with 10XPBS buffer (Lonza™ BioWhittaker™ Phosphate Buffered Saline (10×), BW17-517Q). The antibody supernatant was pre-incubated with protein A agarose for 2 hours. The agarose A-conjugated antibody were applied to the column (BioRad poly-prep chromatography column, #731-1550). The column was washed with low-endotoxin PBS (Lonza™ BioWhittaker™ Dulbecco's Phosphate Buffered Saline (1×) w/o Calcium and Magnesium, BW17512F24). The bound antibody was eluted with elution buffer (ThermoFisher Scientific, Waltham, MA; Elution Buffers, 0.1M Glycin-HCl, pH 2.8, #21004) into Neutralization Buffer (Tris HCl, 1M, BP1757-500). The purified antibody was concentrated and buffer exchanged with PBS, pH 7.0. The antibody concentration was determined by UV absorbance at 280 nm.

To monitor batches during antibody production, attributes of the purified chimeric antibodies, such as purity, isoelectric point (pI) value, amino acid sequence, and N-glycomic profile were assessed (FIGS. 4B-4F). Briefly, purity and pI values were determined by a sodium dodecyl sulfate-polyacrylamide gel electrophoresis (SDS-PAGE) analysis and isoelectric focusing (IEF), respectively. SDS-PAGE or IEF gels were purchased from Bio-Rad Laboratories (Hercules, CA) or ThermoFisher Scientific (Waltham, MA). SDS-PAGE was performed of the 12C chimeric antibody purity under nonreducing and reducing (2-mercaptoethanol) conditions according to the manufacturer's protocol (FIG. 4B). IEF and Coomassie blue staining were also performed according to the manufacturer's protocol. The SDS-PAGE, image acquisition and quantitation of band intensity were performed using Odyssey CLx infrared imaging system (LI-COR Biosciences, Lincoln, NE). FIG. 4B shows SDS-PAGE analysis and FIG. 4C shows IEF analysis of the 12C chimeric antibody.

A peptide mapping comparison for cPD-L1 chimeric antibody (each mAb 12C batch) was also performed to assess amino acid sequence. Briefly, the antibody was enzymatically digested with trypsin on S-trap micro columns from Protifi (Farmingdale, NY) after reduction and alkylation. Peptides were then separated and analyzed by reverse phase liquid chromatography-tandem mass spectrometry (RP-LC-MS/MS) using a Q Exactive HF Hybrid Quadrupole-Orbitrap MS equipped with a Nanospray Flex Ion Source and coupled with a Dionex UltiMate 3000 RSLC Nano System (ThermoFisher Scientific, Waltham, MA). The resultant mass spectrometric data was analyzed using the PEAK PTM workflow in the PEAKS X PRO Studio 10.6 software package from Bioinformatics Solutions Incorporated to map the detected MS1 and MS2 ions to the amino acid sequence of antibody. See Ma et al., PEAKS: powerful software for peptide de novo sequencing by tandem mass spectrometry, Rapid Commun Mass Spectrom 17:2337-2342 (2003). The peptide mapping analysis was performed in the Proteomics core facility at Purdue University (West Lafayette, IN). LC/MS-MS data were used for mapping glycosylation (0.98 Da) of asparagine (N) and glutamine (Q) residues of the mapped antibody sequences.

The sequence coverage of heavy and light chains was 100% (453 of 453 amino acids) and 98.2% (223 of 227 amino acids), respectively, which is a strong positive indicator for SEQ ID NO: 14 being present (FIG. 4D). The chimeric cPD-L1 antibody retained the cPD-L1 binding VH and VL chains of the mouse hybridoma.

Size exclusion chromatography (SEC) analysis was performed on the 12C chimeric antibody to detect antibody aggregates and monomers. The AKTA Pure 150 M (Cytiva, Marlborough, MA) and Superdex 200 Increase 10/300 GL column (Cytiva, Marlborough, MA) were used to analyze antibodies at a flow rate 0.3 ml/min for 135 minutes. Elution was monitored using UV absorption at 280 nm and data were processed by Unicorn 7 software (Cytiva, Marlborough, MA). The SEC analysis was performed in the Molecular Evolution, Protein Engineering, and Production core facility at Purdue University (West Lafayette, IN). FIG. 4E shows SEC analysis results.

In addition, matrix-assisted laser desorption/ionization time-of-flight mass spectrometry profiling (MALDI-TOF MS profiling) of premethylated N-glycans released from PNGase F-treated the 12C chimeric antibody was performed by methods described in Shajahan et al., Glycomic and glycoproteomic analysis of glycoproteins-a tutorial, Anal Bioanal Chem 409:4483-4505 (2017). Briefly, N-glycans of 12C10E4 antibody were released by treating the reduced and alkylated protein with PNGase F. The released N-glycan fractions were then permethylated. The permethylated N-glycans were evaluated by Matrix-assisted laser desorption/ionization-mass spectrometry (MALDI-MS) using the AB SCIEX TOF/TOF 5800 mass spectrometer (Applied Biosystem/MDS Analytical Technologies, Sunnyvale, CA). The structural assignments of the N-glycans were based on molecular weight and followed the principles of the N-glycan bio-synthesis pathway. The carbohydrate analysis was performed at the Complex Carbohydrate Research Center, the University of Georgia (supported by NIH R24GM137782 grant). FIG. 4F shows MALD-MS profiling results.

### Example 6

### Caninized cPD-L1 Chimeric Antibody Binding and Blockade Assays

The caninized cPD-L1 chimeric antibody was assessed using a cell-free cPD-L1/cPD-1 blockade assay pursuant to known protocols. See Li et al. (2018), supra.

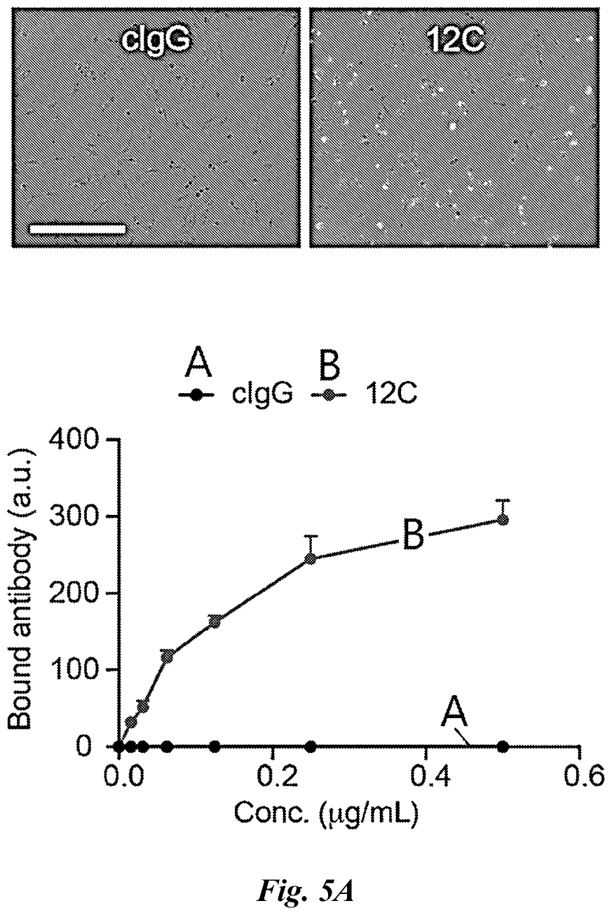

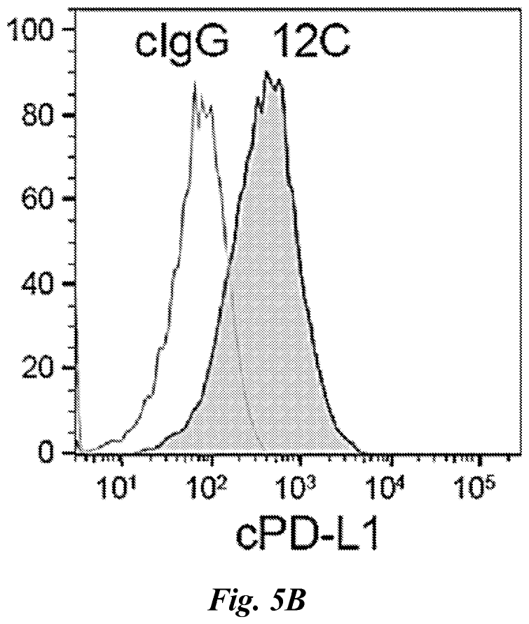

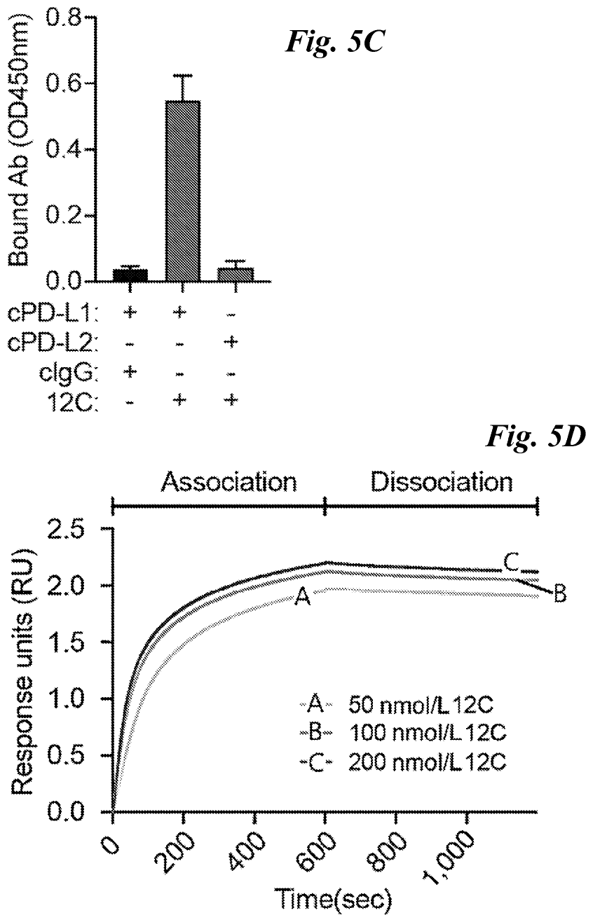

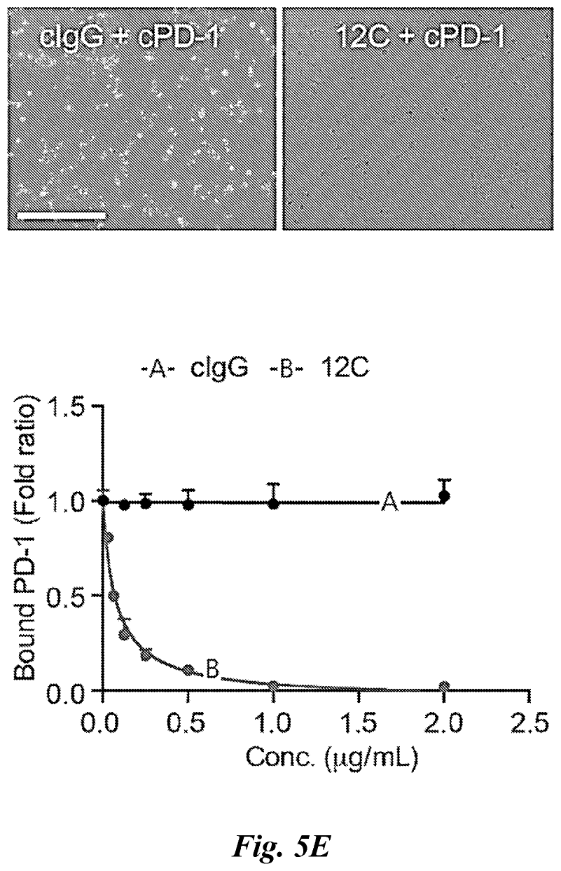

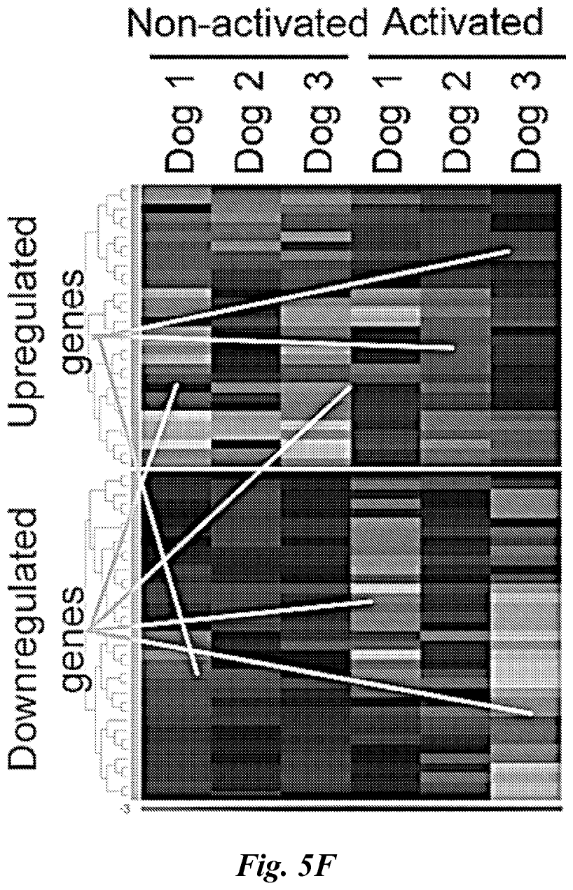

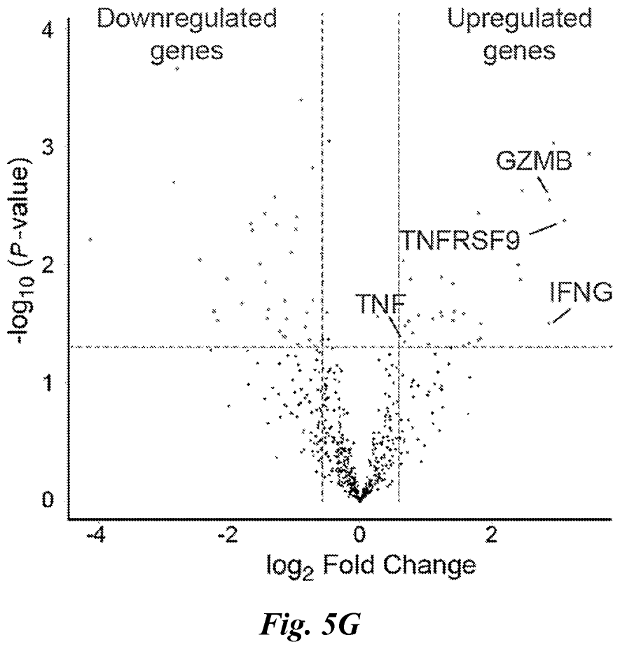

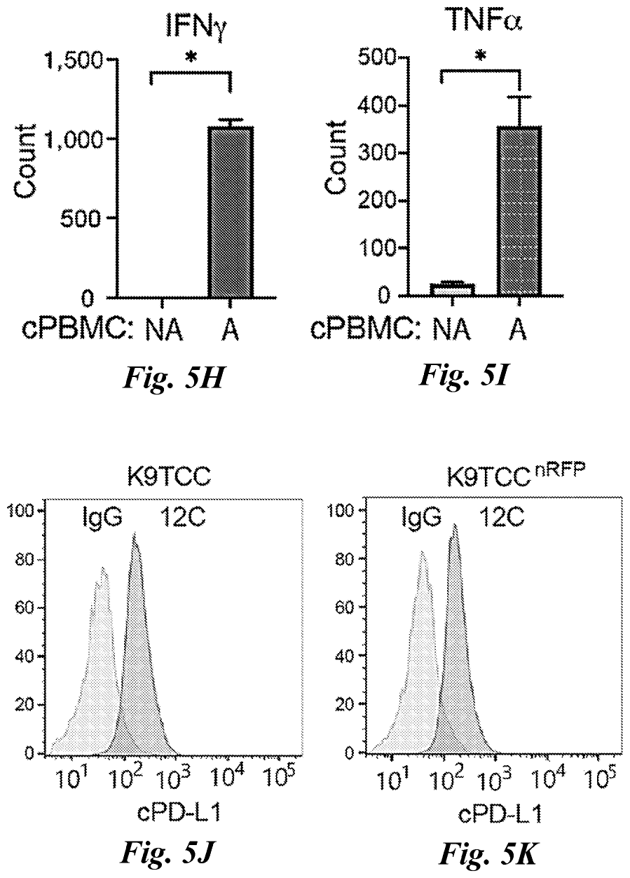

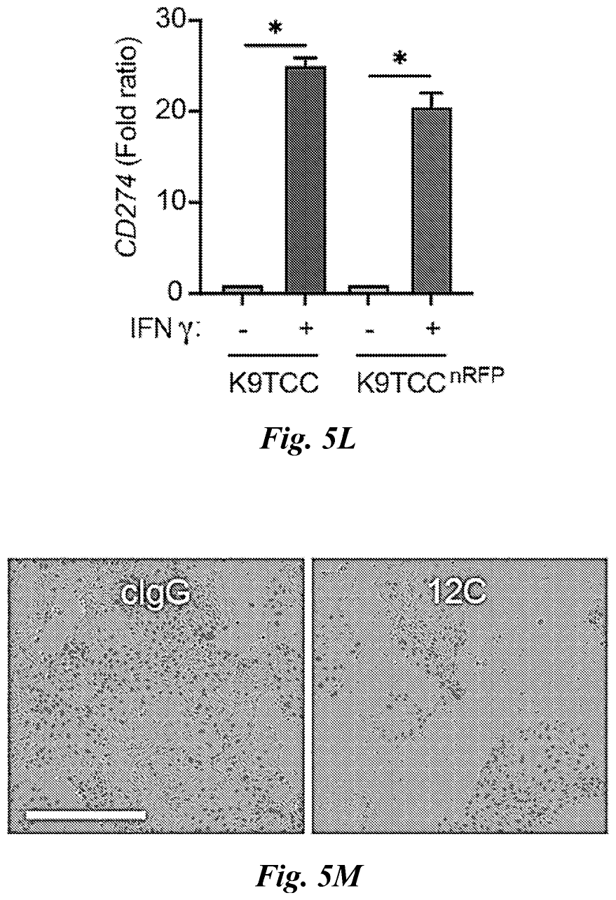

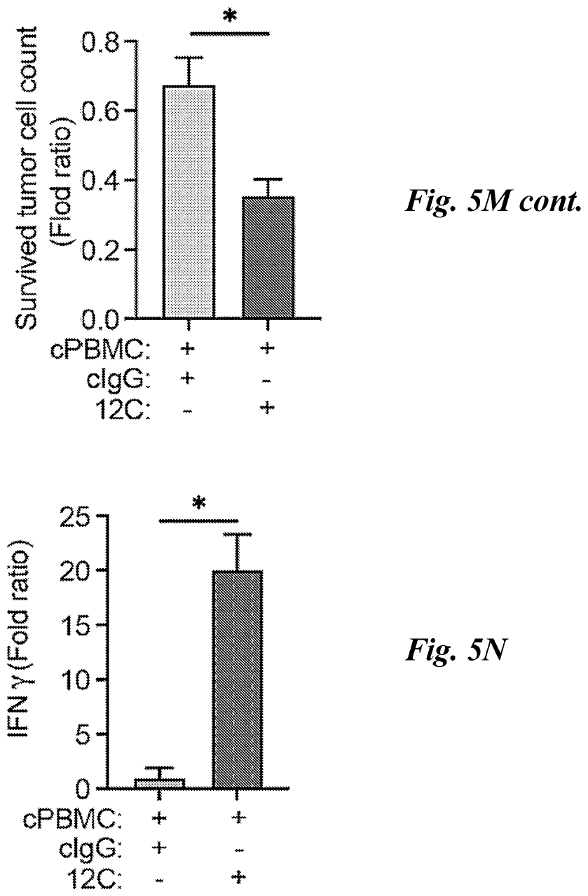

Antibody binding and blockade assays were performed as described in Li et al. (2018), supra. Briefly, to measure PD-L1 protein and PD-L1 antibody interaction, 1×10⁴ BT549^{cPD-L1} cells were seeded per well in 96-well plates. The plates were then incubated with cIgG control (Rockland Immunochemicals, Pottstown, PA) or 12C10E4 antibody, and anti-canine Alexa Fluor 488 dye conjugate (SouthernBiotech, Birmingham, AL). Every hour green fluorescent signal was measured and quantified by IncuCyte S3 (Sartorius, Goettingen, Germany) (FIG. 5A).

To measure PD-1 protein on the cells, 1×10⁴ BT549^{cPD-L1} cells were seeded per well in 96-well plates. The plates were then incubated with cIgG control (Rockland Immunochemicals, Pottstown, PA) or 12C10E4 antibody, cPD-1-hFc protein (human Fc protein conjugated; Sino Biological US, Wayne, PA), and/or anti-human Alexa Fluor 488 dye conjugate (ThermoFisher Scientific, Waltham, MA). Every 3 hours green fluorescent signal was measured and quantified by IncuCyte S3 (Sartorius, Goettingen, Germany) (FIG. 5B). The image analysis was performed according to the manufacturer's protocol.

ELISA based assays were also performed to compare the receptor/ligand and receptor/antibody binding. The 6×His tagged extracellular domain of cPD-L1 proteins were expressed in the ExpiCHO cell system (ThermoFisher Scientific, Waltham, MA) and purified by the Ni-NTA agarose (ThermoFisher Scientific, Waltham, MA) according to the manufacturer's protocol.

For the cPD-L1/cPD-L1 blockade assays, Pierce Ni-NTA coated 96-well plates (ThermoFisher Scientific, Waltham, MA) were coated with canine PD-L1-His proteins and PD-1-Fc proteins (human Fc-protein conjugated) (SinoBiological US, Wayne, PA), and anti-human IgG Fc specific horseradish peroxidase (HRP)-conjugated secondary antibodies (SouthernBiotech, Birmingham, AL) were added, followed by the addition of anti-canine PD-L1, 12C10E4 antibodies. The bound PD-1-Fc protein was quantified by measuring OD450 vale with a Synergy LX multi-mode reader.

The chimeric antibody 12C10E4 bound to the membrane-localized cPD-L1 protein (FIGS. 5A and 5B), but did not recognize cPD-L2 protein (FIG. 5C).

### Example 7

### Binding Affinity (K_{D}) Determination of Caninized cPD-L1 Chimeric Antibody

The binding affinity (K_{D}) of the chimeric cPD-L1/cPD-L1 antibody (12C10E4) for canine PD-L1 was determined by Octet Biolayer interferometry (BLI) using the Octet RED384 system (Sartorius, Bohemia, NY). Briefly, His-tagged cPD-L1 protein was loaded on the Octet NTA biosensor at a concentration of 200 nM. The association step was performed by submerging the sensors in three concentrations of the 12C10E4 antibody (50, 100, 200 nmol/L) in the kinetic buffer. Dissociation was performed and monitored in fresh kinetic buffer. Data were analyzed with Octet Analysis HT software (Sartorius, Bohemia, NY), and the bound cPD-1 proteins were quantified by measuring green fluorescence at the IncuCyte S3.

The K_{D} of the chimeric antibody as determined by Octet was 8.6 nmol/L (FIG. 5D). Similar to the murine 12C antibody obtained from the hybridoma, the 12C chimeric antibody blocked the cPD-L1/cPD-1 interaction (EC₅₀=0.419 μg/ml; FIG. 5E).

### Example 8

### Activation of Peripheral Blood Mononuclear Cells (PBMCs) and Cytokine Measurement

Primary canine PBMCs (cPBMC) were isolated from dog blood using SepMate PBMC isolation tubes (Stemcell Technologies, Inc., Vancouver, B.C., Canada) and Histopaque-1077 (Millipore Sigma, Burlington, MA) per the manufacturer's protocol. The activation of canine T ells by anti-canine CD3 and CD28 antibodies has been well established in previous studies.

Briefly, the canine T cells in PBMCs were activated with 10 ng/mL canine interleukin-2 (IL-2) (10 ng/ml) (Novus Biologicals, Centennial, CO), and co-treated with and without 1 μg/mL anti-canine CD3ε antibody (CA17.2A12 clone, coated; ThermoFisher Scientific, Waltham, MA) and 3 μg/mL (in medium) anti-canine CD28 antibody (1C6 clone; ThermoFisher Scientific, Waltham, MA) for 48 hours. MILLIPLEX Canine Cytokine/Chemokine Magnetic Bead Panel (Sigma-Aldrich, St Louis, MO) was used to multiplex and measure IFNγ, IL-10, and TNFα in these activated canine PBMCs following manufacturer's protocols.

Samples were incubated with the cytokine magnetic beads on shaker for 2 hours followed by incubation with secondary detection antibody provided in the kit. The plate was read on Attune flow cytometer by using the FL2 channel for the reporter (PE channel) and FL4 (APC) channel for classification. For each of the cytokines, 300 beads were measured, and data were collected as forward and side scatter and on a log scale FL2 vs FL4. Concentrations of cytokines were quantified as ng/ml using Cytokine Multiplex Analysis Software (MPLEX, Cytomic Analtyical LLC). The data showed a significant induction in the expression of IFNγ, IL-10 and TNFα with anti-CD3/CD28 and IL2 treatment as compared to IL-2 treatment alone, and this activation protocol was used for the remainder of the studies.

### Example 9

### Gene Expression Analysis of Caninized cPD-L1 Chimeric Antibody

Canine IO Panel (NanoString Technologies, Inc., Seattle, WA) analyses were used to query changes in gene expression upon activation of PBMCs from three healthy pet dogs in Example 8. Activation of the cPBMCs was performed as described previously in Example 8.

RNAs from resting and activated cPBMCs were isolated (RNeasy kit, Qiagen, Germantown, MD) and submitted to Stark Neurosciences Research Institute Biomarker Core, Indiana University School of Medicine (Indiana University, Indianapolis, IN) for detection of modulation of genes upon activation using nCounter® Canine IO Panel (NanoString Technologies, Inc., Seattle, WA).

The Canine IO panel was used to query the changes in about 700 genes. Groupwise analyses of the data were conducted using Rosalind (Rosalind, San Diego, CA) using control cPBMCs (n=3) and compared with activated cells (n=3). There were 65 genes that were differentially expressed when comparing control PBMCs with activated PBMCs (FC≥1.5; P<0.05), including 30 upregulated and 35 downregulated genes.

Data were visualized using heatmap, volcano plot and histogram for specific genes. In the heatmap, each column consists of data from one sample. IFNγ (FIG. 5H) and TNFα (FIG. 5I) concentrations were also analyzed in the activated canine PBMCs.

The canine immune-oncology panel analysis (FIGS. 5F and 5G; Table 1) and analysis of secreted cytokines (IFNγ and TNFα; FIGS. 5H and 5I) demonstrated the activation of canine PBMCs by anti-canine CD3 and CD28 antibodies and canine IL2 treatment.

TABLE 1
Genes altered in the activated cPBMCs from the
nCounter canine immune-oncology panel analysis.

| | | | Log2 | | | Ave. |
|---|---|---|---|---|---|---|
| | | Fold | Fold | | | Log2 |
| Name | Description | Change | Change | p-Value | p-Adj | Exp. |
| S100A4 | S100 calcium binding protein A4 | 2.70 | 1.43 | 0.02 | 0.34 | 11.23 |
| IFNG | interferon gamma | 7.32 | 2.87 | 0.03 | 0.35 | 8.35 |
| IL2RA | interleukin 2 receptor, alpha | 1.84 | 0.88 | 0.03 | 0.34 | 9.80 |
| TNF | tumor necrosis factor | 1.60 | 0.67 | 0.04 | 0.39 | 8.56 |
| CSF2 | colony stimulating factor 2 | 2.98 | 1.57 | 0.03 | 0.34 | 7.21 |
| IL12A | interleukin 12A | 1.61 | 0.68 | 0.03 | 0.35 | 6.15 |
| BIRC5 | baculoviral IAP repeat | 2.97 | 1.57 | 0.05 | 0.40 | 6.32 |
| | containing 5 | | | | | |
| BRCA2 | breast cancer 2, early onset | 5.41 | 2.44 | 0.01 | 0.26 | 5.35 |
| CDKN2C | cyclin-dependent kinase | 2.36 | 1.24 | 0.01 | 0.26 | 6.92 |
| | inhibitor 2C | | | | | |
| PCNA | proliferating cell nuclear | 1.71 | 0.77 | 0.01 | 0.26 | 9.32 |
| | antigen | | | | | |
| C1QA | complement component 1, q | 3.15 | 1.66 | 0.05 | 0.39 | 5.66 |
| | subcomponent, A chain | | | | | |
| CXCL10 | chemokine (C-X-C motif) | 2.66 | 1.41 | 0.03 | 0.34 | 14.16 |
| | ligand 10 | | | | | |
| ILF3 | interleukin enhancer binding | 1.67 | 0.74 | 0.03 | 0.34 | 7.37 |
| | factor 3, 90 kDa | | | | | |
| C1S | complement component 1, s | 3.57 | 1.84 | 0.03 | 0.35 | 6.11 |
| | subcomponent | | | | | |
| LAG3 | lymphocyte-activation gene 3 | 3.56 | 1.83 | 0.04 | 0.39 | 6.69 |
| GZMA | granzyme A | 11.23 | 3.49 | 0.00 | 0.13 | 8.74 |
| BST1 | bone marrow stromal cell | 2.05 | 1.04 | 0.05 | 0.39 | 5.44 |
| | antigen 1 | | | | | |
| PRF1 | perforin 1 | 2.34 | 1.23 | 0.03 | 0.34 | 7.25 |
| CD3G | CD3g molecule, gamma | 1.58 | 0.66 | 0.01 | 0.23 | 9.76 |
| LOC490356 | cytokine SCM-1 beta-like | 7.70 | 2.95 | 0.00 | 0.13 | 8.75 |
| LOC490629 | transmembrane protease serine | 2.38 | 1.25 | 0.03 | 0.34 | 6.47 |
| | 9-like | | | | | |
| GZMB | granzyme B | 7.38 | 2.88 | 0.00 | 0.16 | 8.84 |
| TBX21 | T-box 21 | 2.66 | 1.41 | 0.01 | 0.26 | 8.23 |
| LTA | lymphotoxin alpha | 2.15 | 1.11 | 0.03 | 0.34 | 6.70 |
| TYMS | thymidylate synthetase | 1.74 | 0.80 | 0.04 | 0.38 | 7.87 |
| KLRB1 | killer cell lectin-like receptor | 3.49 | 1.80 | 0.00 | 0.16 | 7.55 |
| | subfamily B, member 1 | | | | | |
| MKI67 | antigen identified by | 5.30 | 2.40 | 0.01 | 0.23 | 6.40 |
| | monoclonal antibody Ki-67 | | | | | |
| CDK1 | cyclin-dependent kinase 1 | 3.49 | 1.80 | 0.04 | 0.39 | 6.07 |
| NKG7 | natural killer cell group 7 | 5.53 | 2.47 | 0.00 | 0.16 | 7.34 |
| | sequence | | | | | |
| IL13RA1 | interleukin 13 receptor, alpha 1 | −1.67 | −0.74 | 0.00 | 0.15 | 8.16 |
| TNFRSF1A | tumor necrosis factor receptor | −1.53 | −0.61 | 0.04 | 0.39 | 8.72 |
| | superfamily, member 1A | | | | | |
| IL1RN | interleukin 1 receptor | −1.95 | −0.96 | 0.05 | 0.39 | 9.86 |
| | antagonist | | | | | |
| PDPN | podoplanin | −4.54 | −2.18 | 0.03 | 0.34 | 6.74 |
| ADORA2A | adenosine A2a receptor | −1.96 | −0.97 | 0.00 | 0.16 | 8.40 |
| IL1B | interleukin 1, beta | −3.51 | −1.81 | 0.02 | 0.34 | 7.40 |
| CD36 | CD36 molecule | −3.15 | −1.66 | 0.01 | 0.16 | 5.36 |
| PLAUR | plasminogen activator, | −2.49 | −1.31 | 0.00 | 0.16 | 8.56 |
| | urokinase receptor | | | | | |
| LRP1 | low density lipoprotein | −2.44 | −1.29 | 0.00 | 0.16 | 7.54 |
| | receptor-related protein 1 | | | | | |
| CSF2RB | colony stimulating factor 2 | −2.35 | −1.23 | 0.04 | 0.38 | 7.54 |
| | receptor, beta | | | | | |
| NCF4 | neutrophil cytosolic factor 4 | −2.19 | −1.13 | 0.03 | 0.34 | 6.64 |
| TLR3 | toll-like receptor 3 | −4.11 | −2.04 | 0.01 | 0.26 | 4.99 |
| SIGLEC1 | sialic acid binding Ig-like | −2.66 | −1.41 | 0.02 | 0.34 | 6.73 |
| | lectin 1 | | | | | |
| HCK | hemopoietic cell kinase | −2.74 | −1.46 | 0.01 | 0.26 | 7.30 |
| SDC4 | syndecan 4 | −2.23 | −1.16 | 0.02 | 0.34 | 10.96 |
| CMKLR1 | chemokine-like receptor 1 | −2.68 | −1.42 | 0.03 | 0.34 | 6.51 |
| ITGA5 | integrin, alpha 5 | −1.66 | −0.73 | 0.02 | 0.34 | 8.59 |
| LTBR | lymphotoxin beta receptor | −2.09 | −1.06 | 0.01 | 0.22 | 6.08 |
| THBS1 | thrombospondin 1 | −5.47 | −2.45 | 0.01 | 0.23 | 5.52 |
| IL1RAP | interleukin 1 receptor accessory | −2.21 | −1.14 | 0.04 | 0.39 | 7.27 |
| | protein | | | | | |
| CSF1R | colony stimulating factor 1 | −3.20 | −1.68 | 0.00 | 0.16 | 6.90 |
| | receptor | | | | | |
| ITGAM | integrin, alpha M | −6.96 | −2.80 | 0.00 | 0.12 | 7.36 |
| CXCR3 | chemokine (C-X-C motif) | −1.77 | −0.82 | 0.03 | 0.34 | 8.00 |
| | receptor 3 | | | | | |
| TNFRSF9 | tumor necrosis factor receptor | 8.65 | 3.11 | 0.00 | 0.16 | 6.75 |
| | superfamily, member 9 | | | | | |
| CD55 | CD55 molecule, decay | −1.80 | −0.85 | 0.03 | 0.36 | 7.27 |
| | accelerating factor for | | | | | |
| | complement | | | | | |
| SERPINB2 | serpin peptidase inhibitor, clade | −17.43 | −4.12 | 0.01 | 0.19 | 6.33 |
| | B (ovalbumin), member 2 | | | | | |
| AMICA1 | adhesion molecule, interacts | −1.63 | −0.70 | 0.05 | 0.40 | 8.63 |
| | with CXADR antigen 1 | | | | | |
| FADD | Fas (TNFRSF6)-associated via | −1.51 | −0.60 | 0.01 | 0.22 | 8.23 |
| | death domain | | | | | |
| CD9 | CD9 molecule | −2.76 | −1.47 | 0.00 | 0.16 | 9.46 |
| FCRL2 | Fc receptor-like 2 | −2.89 | −1.53 | 0.01 | 0.23 | 7.50 |
| IL6R | interleukin 6 receptor | −1.98 | −0.99 | 0.01 | 0.16 | 6.12 |
| CEACAM1 | carcinoembryonic antigen- | −1.72 | −0.79 | 0.04 | 0.39 | 7.89 |
| | related cell adhesion molecule 1 | | | | | |
| PYCARD | PYD and CARD domain | −2.27 | −1.18 | 0.04 | 0.39 | 6.36 |
| | containing | | | | | |
| LOC102153988 | uncharacterized LOC102153988 | −4.71 | −2.24 | 0.02 | 0.34 | 8.47 |
| LOC102154078 | uncharacterized LOC102154078 | −7.20 | −2.85 | 0.00 | 0.16 | 7.13 |
| VSIR | | −1.89 | −0.92 | 0.00 | 0.12 | 9.24 |

### Example 10

### Caninized cPD-L1 Chimeric Antibody Enhances T Cell-Mediated Tumor Cell Killing

To demonstrate immune checkpoint inhibition of the 12C chimeric antibody and analyze the killing of tumor cells by T-cell inactivation, a tumor cell killing assay was performed pursuant to known protocols (see Li et al. (2016), supra) on an ex vivo canine system in which cPD-L1-positive canine bladder cells (K9TCC) were co-cultured with activated canine PBMCs. To quantify the number of surviving or dead tumor cells in a tumor cell killing assay, a K9TCCIRFP cells expressing nuclear-restricted red fluorescent protein (RFP) were established. Expression of endogenous cPD-L1 protein and mRNA in both K9TCC parental and K9TCC^{nRFP} cells upon IFNγ treatment was confirmed (FIGS. 5J-5L). These activated canine PBMCs and K9TCC^{nRFP} cells were then used to perform a tumor cell killing assay.

Briefly, the K9TCCRFP cells were co-cultured with activated cPBMCs in DMEM/F12 with 10% FBS. cPBMCs were activated via incubation with 100 ng anti-canine CD3ε antibody (see Example 8) (CA17.2A12 clone, ThermoFisher Scientific, Waltham, MA)) and 10 ng/ml canine interleukin-2 (IL-2) (Novus Biologicals, Littleton, CO) in DMEM/F12 with 10% FBS. The primary cPBMCs were isolated from dog blood using SepMate PBMC isolation tubes (Stemcell Technologies, Cambridge, MA) and Histopaque-1077 (Sigma-Aldrich, St. Louis, MO) per the manufacturer's protocol.

The live tumor cell count at 72 hours is shown in the bar graph of FIG. 5J. After 96 hours, RFP signals were measured as survived tumor cells, and the expression of IFNγ, IL10, and TNFα in the supernatant of the co-cultured cells was measured by MILLIPLEX Canine Cytokine/Chemokine Magnetic Bead Panel according to manufacturer's protocols.

Although the PBMC and tumor cells were from different dogs, and thus the dog lymphocyte antigen (DLA) was not matched between the cPBMCs and K9TCC cells, the 12C chimeric antibody enhanced tumor cell killing activity and IFNγ secretion (FIGS. 5M and 5N).

### Example 11

### Treatment of Laboratory Dogs with 12C10E4 Chimeric Antibody

A single-dose pilot study to assess initial safety and pharmacokinetic parameters was performed in six laboratory beagles approximately 12-15 months old, with male and female dogs included. The dogs were housed and evaluated in the Pre-Clinical Research Laboratory, College of Veterinary Medicine, Purdue University, West Lafayette, IN. The 12C10E4 cPD-L1 chimeric antibody for the laboratory dog study was produced in the Molecular Evolution, Protein Engineering, and Production Facility at Purdue University (West Lafayette, IN) as described above. The antibody solution was Mycoplasma free and contained less than 0.5 EU endotoxin/mg antibody (consistent with the endotoxin limit for human PD-L1 antibody solutions).

After being acclimated to the facility, the dogs were treated with the 12C10E4 cPD-L1 chimera antibody diluted in sterile water for intravenous (IV) administration (6 ml/kg body weight total volume) and administered through an IV catheter over 1 hour. Six dogs were treated and received 2 mg/kg or 5 mg/kg antibody.

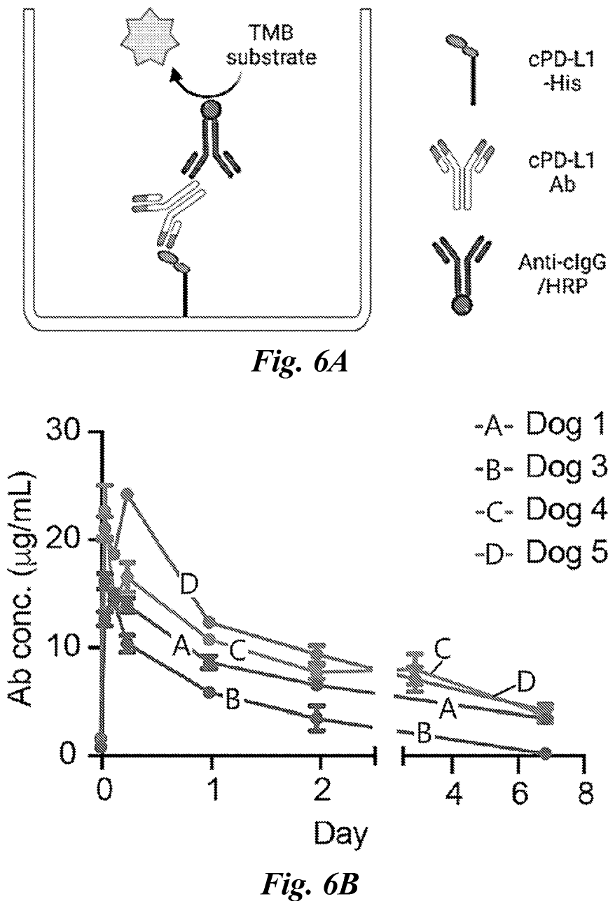

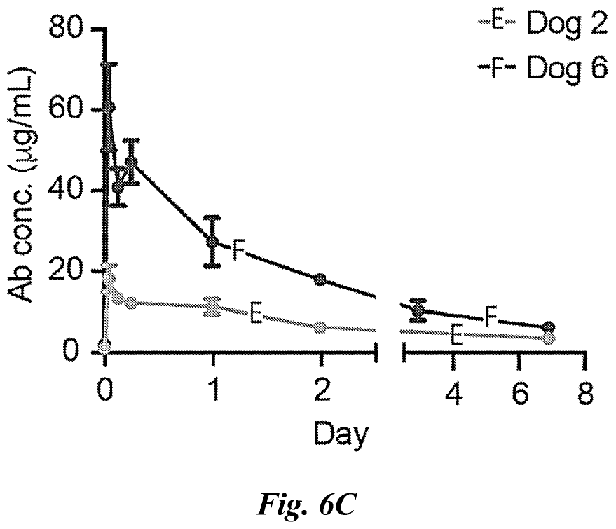

Blood was collected for pharmacokinetic analyses at 1, 6, 24, 48, and 72 hours after the start of antibody administration, and then once weekly for 4 weeks. Monitoring for adverse events included physical exam before and during treatment, then twice daily for 7 days, and then weekly for 4 weeks; daily observation for 4 weeks; and complete blood count (CBC), serum biochemistry panel (including, without limitation, measurement of the concentration of the 12C10E4 chimeric antibody in the 12C10E4 antibody-treated dogs' serum (FIG. 6A)), and urinalysis before treatment and weekly for 4 weeks. Additional tests were planned specific to any adverse events observed. Adverse events were categorized using Veterinary Cooperative Oncology Group (VCOG) criteria. See VCOG, Common terminology criteria for adverse events (VCOG-CTCAE) following chemotherapy or biological antineoplastic therapy in dogs and cats v1.1, Vet Comp Oncol 14:417-446 (2016).

The 12C10E4 chimeric antibody was well tolerated and had a half-life of about 3 days (FIGS. 6B and 6C; Table 2). The half-life of the cPD-L1 antibody was shorter than that for human checkpoint inhibitors and indicated that weekly dosing could be appropriate for dogs. The possible infusion reaction in one dog resolved without intervention. There was good antibody tolerability of the antibody in this single-dose study in the lab dogs and body weight was maintained (Table 2). Nonspecific changes such as a slight reduction in monocyte count reduction and a slight increase in CO₂ and gamma-glutamyl transferase (GGT) were transient and resolved without intervention (Table 2).

TABLE 2
Summary of potential adverse events with the initial
cPD-L1 antibody administration to laboratory dogs.

| Parameter | Result |
|---|---|
| Dogs | 3 intact female, 3 intact male beagles, |
| | 12-15 months of age |
| Antibody dosing | Administered 2 mg/kg (4 dogs) or 5 mg/kg (2 dogs) |
| | antibody given by slow intravenous infusion over 1 |
| | hour |
| Abnormalities in | Slightly low monocyte count (0.09 × 10³/μL, |
| complete blood | reference range 0.15-1.35 × 10³/μL) at 1 week post |
| counts or serum | treatment; normalized by 2 weeks post treatment (1 |
| biochemical | dog, 2 mg/kg cPD-L1 antibody). |
| profiles | Slightly high CO₂ (25 mmol/L, reference range |
| | 13-24 mmol/L) at 1 week post treatment normalized |
| | by 2 weeks post treatment in 1 dog and by 4 weeks |
| | after antibody treatment in a second dog (1 dog, |
| | 2 mg/kg cPDL-1 antibody and the second dog 5 |
| | mg/kg cPDL-1 antibody). |
| | Slightly high GGT (18 IU/L, reference range 5-16 |
| | IU/L) at 1 week post treatment; normalized by 2 |
| | weeks post treatment (1 dog, 5 mg/kg cPD-L1 |
| | antibody). |
| | Slightly high cholesterol (306 mg/dL, reference |
| | range 124-301 mg/dL) at 1 week after treatment; |
| | normalized by 6 weeks after treatment (1 dog, |
| | 5 mg/kg cPD-L1 antibody). |
| Other | Possible infusion reaction in 1 dog who experienced |
| observations | weakness, pale mucous membranes, and bradycardia |
| | (reduced heart rate) that started 5 minutes after the |
| | completion of the antibody infusion. The dog |
| | returned to normal within 10 minutes with no |
| | intervention (1 dog, 5 mg/kg cPD-L1 antibody). |
| | Mildly decreased appetite the day of treatment in 3 |
| | dogs (2 dogs, 2 mg/kg cPD-L1 antibody; 1 dog 5 |
| | mg/kg cPD-L1 antibody). |
| | Other than these findings, the dogs remained bright, |
| | alert, and active, maintained body weight, and had |
| | normal temperature/pulse/respiration. |

### General and Certain Definitions

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

In the following description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary, but remain applicable in view of the data provided herein.

Various techniques and mechanisms of the present disclosure will sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, wherever feasible and convenient, like reference numerals are used in the figures and the description to refer to the same or like parts or steps. The drawings are in a simplified form and not to precise scale. It is understood that the disclosure is presented in this manner merely for explanatory purposes and the principles and embodiments described herein can be applied to antibodies and/or composition components that have configurations other than as specifically described herein. Indeed, it is expressly contemplated that the components of the composition and compounds of the present disclosure may be tailored in furtherance of the desired application thereof.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein. Additionally, as used in this specification and the appended claims, the singular forms “a”, “an” and “the” include plural referents unless the content clearly dictates otherwise. Thus, for example, where a compound/composition is substituted with “an” alkyl or aryl, the compound/composition is optionally substituted with at least one alkyl and/or at least one aryl.

When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range can vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

“Substantially” can allow for a degree of variability in a value or range, for example, within 90%, within 95%, or within 99% of a stated value or of a stated limit of a range.

The terms “sequence identity” or “percent identity,” in the context of two or more polypeptide sequences, refer to two or more sequences or subsequences that are the same or have a specified percentage of peptides that are the same (i.e. about 60% identity, preferably 65%, 70%, 75%, 80%, 85%, 90%, 91%, 92%, 93%, 94%, 95%, 96%, 97%, 98%, 99%, or higher identity over a specified region when compared and aligned for maximum correspondence over a comparison window or designated region such as a targeted gene) as measured using sequence comparison algorithms known in the art, or by manual alignment and visual inspection. Such sequences are then said to be “substantially identical.” In other words, identity exists over one or more regions of the overall sequence as long as the general shape and structure of the molecule, and hydrogen bond(s) where appropriate, are maintained such that it substantially fits into the targeted binding site and functions as an agonist thereto.

The term “fully canine antibody” refers to an antibody that comprises canine immunoglobulin protein sequences only. A fully canine antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully canine antibody may contain rat carbohydrate chains if produced in a rat, in a rat cell, or in a hybridoma derived from a rat cell. Similarly, “rat antibody” refers to an antibody that comprises rat immunoglobulin sequences only.

In certain embodiments, the compounds, compositions and methods of the present disclosure are useful for the prevention and/or treatment of cancer. In certain embodiments, the compounds and/or compositions provided are also useful for the treatment of cancer. In certain embodiments, the compounds provided herein are provided or used alone, in conjunction with a targeting agent, and/or in a combination therapy with other interventions such as cytokine-based and other immunotherapies.

1. A caninized antibody or antigen binding fragment thereof that binds programmed death ligand 1 (PD-L1) in a canine subject with specificity, wherein the caninized antibody or antigen binding fragment thereof is encoded by the nucleotide sequence comprising at least 80% sequence identity to SEQ ID NOS: 2 and/or 4.

2. (canceled)

3. The antibody or antigen binding fragment of claim 1, comprising one or more complementarity determining regions (CDRs), each CDR comprising at least 80% sequence identity to AAS, SEQ ID NO: 9, and/or SEQ ID NO: 10.

4. (canceled)

5. The antibody or antigen binding fragment of claim 1, comprising CDRs independently comprising at least 80% sequence identity to SEQ ID NO: 15, SEQ ID NO: 16, and/or SEQ ID NO: 11.

6. (canceled)

7. The antibody or antigen binding fragment of claim 1, wherein the antibody or antigen binding fragment thereof is completely or incompletely caninized, or wherein the antibody or antigen binding fragment is a chimeric form of a caninized antibody or antigen binding fragment, or wherein the antibody or antigen binding fragment comprises a caninized murine PD-1 antibody or antigen binding fragment thereof.

8.-9. (canceled)

10. A method for treating cancer in a canine subject comprising administering to a canine subject a therapeutically effective amount of the antibody or antigen binding fragment of claim 1.

11. The method of claim 10, wherein the caninized antibody or antigen binding fragment thereof is encoded by a nucleotide sequence comprising at least 80% sequence identity to SEQ ID NOS: 2 and/or 4.

12. The method of claim 10, wherein the caninized antibody or antigen binding fragment comprises one or more complementarity determining regions (CDRs) independently comprising at least 80% sequence identity to AAS, SEQ ID NO: 9, and/or SEQ ID NO: 10.

13. (canceled)

14. The method of claim 10, wherein the caninized antibody or antigen binding fragment comprises one or more CDRs comprising at least 80% sequence identity to SEQ ID NO:
15, SEQ ID NO: 16, and/or SEQ ID NO: 11.

15. (canceled)

16. The method of claim 10, wherein the antibody or antigen binding fragment thereof is formulated into a pharmaceutical composition.

17. The method of claim 10, wherein the therapeutically effective amount is from about 2 mg/kg of subject body weight to about 5 mg/kg of subject body weight.

18.-20. (canceled)

21. The method of claim 10, wherein the cancer is invasive urothelial carcinoma.

22. A pharmaceutical composition comprising an antibody or antigen binding fragment thereof and a pharmaceutically acceptable excipient, wherein the antibody or antigen binding fragment thereof is encoded by a nucleotide sequence comprising at least 80% sequence identity to SEQ ID NOS: 2 and/or 4.

23.-28. (canceled)

29. The pharmaceutical composition of claim 22, wherein the antibody or antigen binding fragment thereof further comprises one or more complementarity determining regions (CDRs), each CDR comprising at least 80% sequence identity to AAS, SEQ ID NO: 9, and/or SEQ ID NO: 10.

30. (canceled)

31. The pharmaceutical composition of claim 22, wherein the antibody or antigen binding fragment thereof further comprises one or more complementarity determining regions (CDRs), each CDR comprising at least 80% sequence identity to SEQ ID NO: 15, SEQ ID NO: 16, and/or SEQ ID NO: 11.

32.-36. (canceled)

37. The pharmaceutical composition of claim 22, wherein the pharmaceutical composition of claim is used in the preparation of a medicament for treating cancer in a canine subject.

38. The pharmaceutical composition of claim 29, wherein the pharmaceutical composition is used in the preparation of a medicament for treating cancer in a canine subject.

39. The pharmaceutical composition of claim 31, wherein the pharmaceutical composition is used in the preparation of a medicament for treating cancer in a canine subject.

40. The antibody or antigen binding fragment of claim 3, wherein the heavy chain of the antibody comprises SEQ ID NO: 3.

41. The antibody or antigen binding fragment of claim 3, comprising CDRs independently comprising at least 80% sequence identity to SEQ ID NO: 15, SEQ ID NO: 16, and/or SEQ ID NO: 11.
